(19) United States
(12) Patent Application Publication
(10) Pub. No.: US 2006/0173257 A1
(43) Pub. Date: Aug. 3, 2006

(54) Sleep evaluation method, sleep evaluation system, operation program for sleep evaluation system, pulse oximeter, and sleep support system

(75) Inventors: Yoshiroh Nagai, Nishinomiya-shi (JP); Kazumi Kitajima, Higashiosaka-shi (JP)

Correspondence Address: SIDLEY AUSTIN LLP, 717 NORTH HARWOOD, SUITE 3400, DALLAS, TX, 75201, US

(73) Assignee: KONICA MINOLTA SENSING, INC.

(21) Appl. No.: 11/341,326

(22) Filed: Jan. 27, 2006

(30) Foreign Application Priority Data
Jan. 31, 2005 (JP) 2005-23674

(51) Int. Cl. A61B 5/00; A61G 7/00

(52) U.S. Cl. 600/323; 5/600

(57) ABSTRACT

Measurement is made about a necessary evaluation parameter of a subject, the parameter being variable due to sleep apnea of the subject. A body position of the subject is detected in terms of angle information. The parameter measurement and the body angle detection are executed at a predetermined sampling frequency. These data are stored in a storage. A sleep evaluation system includes: an evaluation parameter detector for measuring an evaluation parameter of a subject, a body position detector for detecting a body position of the subject in terms of angle information; a storage for storing measurement data acquired by the evaluation parameter detector and by the body position detector therein; and a controller for causing the evaluation parameter detector to measure the evaluation parameter, and causing the body position detector to measure a body angle of the subject at a predetermined sampling frequency to store the measurement data in the storage.

This application is based on Japanese Patent Application No. 2005-23674 filed on Jan. 31, 2005, the contents of which are hereby incorporated by reference.

### BACKGROUND OF THE INVENTION

1. Field of the Invention

The present invention relates to a method and a system for evaluating sleep of subjects in diagnosing sleep apnea syndrome (hereinafter, called as “SAS”), and more particularly to a sleep evaluation method and a sleep evaluation system capable of accurately obtaining a correlation between apnea and body position of a subject in sleep.

2. Description of the Related Art

SAS, which may cause various diseases resulting from apnea or low respiration in sleep, have recently raised social issues, considering a possibility that SAS may not only cause high blood pressures, cerebrovascular disorders, or ischemic heart diseases, but also may lower labor productivity or induce severe work-related accidents due to daytime sleepiness. There is proposed an approach of measuring a variation in oxygen saturation in an arterial blood (hereinafter, called as “SpO₂” or “blood oxygen saturation”) of a subject in sleep, as a process for screening SAS in light of a fact that oxygen is not supplied to the arterial blood in apnea, and the blood oxygen saturation is resultantly lowered.

Conventionally, there is known a pulse oximeter for measuring the blood oxygen saturation. In the pulse oximeter, a variation in blood oxygen saturation with time is detected by detachably attaching a probe provided with a light emitter and a light detector to a subject's finger, causing the light emitter to project light toward the subject's finger, detecting a variation in light amount passed through the finger in terms of a pulse signal, and processing the measurement result obtained every second interval by a moving-average method. The degree of severity of SAS is determined by integrating the number of peaks where the blood oxygen saturation is lowered, which is supposed to be caused by apnea. An example of indexes for judging the degree of severity of SAS is an oxygen desaturation index (ODI), which represents the number of peaks where blood oxygen saturation is lowered per hour. ODI can be measured with use of the pulse oximeter. Use of the pulse oximeter is simple, because a subject is merely required to attach the probe to his or her finger. Accordingly, it can be said that ODI is a useful index with which a subject i.e. a SAS patient is allowed to easily measure the degree of severity of SAS at home in a condition close to his or her ordinary sleep. Medical institutes typically use apnea hypopnea index (AHI), which represents the number of apneustic respirations or low respirations per hour. AHI is obtained by using polysomnography (PSG) for detecting various evaluation parameters, in addition to the blood oxygen saturation, such as electroencephalogram, airflow by mouth/nasal breathing, snoring sounds, movements of chest/abdomen, and body position.

It is known that there is a certain causal relation or correlation between apnea and body position of a SAS patient in sleep. Specifically, since the soft palate or posterior part of tongue of a SAS patient is likely to block the pharyngeal cavity while the patient lies in a supine (face-up) position, apnea may likely to be caused due to blockage of the respiratory passage. On the other hand, since blockage of the respiratory passage is unlikely to occur while the patient lies in a lateral decubitus position or in a prone (face-down) position, the patient may relatively unlikely to suffer from apnea when the patient lies in such a position. Since there is a significant correlation between apnea and body position of a SAS patient, the PSG features the body position as one of the evaluation parameters.

The body position, which is regarded as one of the evaluation parameters by the conventional PSG, is roughly classified into four directions, namely, a supine position, a right lateral decubitus position, a left lateral decubitus position, and a prone position; or nine positions, namely, a mid position between the supine position and the right lateral decubitus position, a mid position between the right lateral decubitus position and the prone position, a mid position between the prone position and the left lateral decubitus position, a mid position between the left lateral decubitus position and the supine position, and a seated position in addition to the above four positions. Accordingly, the conventional PSG has failed to provide sufficient information relating to subtle movements of a subject, which obstructs a user including the subject from accurately recognizing the correlation between apnea and body position. Also, the correlation between apnea and body position differs among individuals. However, the conventional PSG has failed to provide information relating to individual differences, which obstructs a medical staff from accurately determining an optimal approach for treating individual subjects e.g. suggesting a recommended body position in which the subject should lie in sleep.

FIG. 29 is a graph showing a relation between change of SpO₂ with time, and actual change of the body position. The graph shows that SpO₂ is lowered twice i:e. at a time duration tq1 and a time duration tq2. In this case, according to the conventional PSG, merely the roughly classified body positions as mentioned above are acquired as the body position parameter. Accordingly, the conventional PSG judges that the body positions at the time durations tq1 and tq2 when SpO₂ is lowered are “supine position”, as well as the time between the time durations tq1 and tq2 when SpO₂ shows a normal value, which disables the medical staff to properly evaluate the correlation between apnea and body position. Obviously, apnea shows dependence on body position that SpO₂ is lowered when the body position is in a position corresponding to a time duration tq31, and in a position corresponding to a time duration tq33, and that SpO₂ is not lowered when the body position is in a position corresponding to a time duration tq32. However, the conventional PSG has failed to provide an evaluation which precisely reflects the dependence.

Also, as shown in FIG. 30, for instance, using a PSG capable of evaluating the body position in eight different positions enables to evaluate the correlation between apnea and body position to some details. However, it is likely that data may be fluctuated in a time duration indicated by a circle td if the body position is a threshold position of judging a change of the body position.

### SUMMARY OF THE INVENTION

It is an object of the present invention to provide a sleep evaluation method, a sleep evaluation system, an operation program for the sleep evaluation system, a pulse oximeter, and a sleep support system that enable to accurately obtain a correlation between body position and apnea of a subject in terms of data.

According to an aspect of the invention, measurement is made about a necessary evaluation parameter of a subject, the parameter being variable due to sleep apnea of the subject. A body position of the subject is detected in terms of angle information. The parameter measurement and the body angle detection are executed at a predetermined sampling frequency. These data are stored in a storage.

These and other objects, features and advantages of the present invention will become more apparent upon reading of the following detailed description along with the accompanying drawings.

### BRIEF DESCRIPTION OF THE DRAWINGS

FIG. 1 is a block diagram schematically showing an entire arrangement of a sleep evaluation system in accordance with an embodiment of the invention.

FIG. 2 is an illustration showing an example of a sleep evaluation system having a certain hardware construction.

FIG. 3 is an illustration for describing a manner as to how a pulse oximeter serving as an evaluation parameter detector is attached to a subject.

FIG. 4 is a circuit diagram schematically showing a circuit configuration of the pulse oximeter.

FIGS. 5A through 5C are illustrations showing a three-axis acceleration sensor using a piezoresistor, as an example of a three-axis acceleration sensor, wherein FIG. 5A is a perspective view of the three-axis acceleration sensor, FIG. 5B is a top plan view thereof, and FIG. 5C is a cross-sectional view taken along the line a-a in FIG. 5B.

FIG. 6A is an illustration schematically showing a beam model deformed in X-axis direction and Y-axis direction.

FIG. 6B is a circuit diagram schematically showing a bridge circuit for detecting a voltage variation representing the deformation of the beam model shown in FIG. 6A.

FIG. 7A is an illustration schematically showing a beam model deformed in Z-axis direction.

FIG. 7B is a circuit diagram schematically showing a bridge circuit for detecting a voltage variation representing the deformation of the beam model shown in FIG. 7A.

FIG. 8 is an illustration for explaining a principle as to how tilt angles of X-axis, Y-axis, and Z-axis are defined in expressing the position of the acceleration sensor.

FIG. 9 is a perspective view showing a correlation between the X-axis, Y-axis, and Z-axis of the acceleration sensor shown in FIG. 8, and a lying position of a subject.

FIG. 10 is an illustration showing a state that the three-axis acceleration sensor is incorporated in a main body of the pulse oximeter.

FIG. 11 is a block diagram showing an arrangement of electrical functions of the pulse oximeter.

FIG. 12 is a block diagram showing an arrangement of electrical functions of a personal computer main body, specifically, an analyzer and a processor.

FIG. 13 is a graph showing an example of an SpO₂ curve.

FIG. 14 is an illustration schematically showing indexes for detecting a Dip in an SpO₂ curve.

FIG. 15 is a time chart briefly describing an example of data relating to a change of body angle with time.

FIG. 16 is a time chart showing an example of composite data generated by combining an SpO₂ curve and a change of body angle with time.

FIG. 17 is a graph showing a histogram of angle-based ODI with respect to a subject A to know a body angle range of the subject A where apnea is observed.

FIG. 18 is a graph showing a histogram of angle-based ODI with respect to a subject B to know a body angle range of the subject B where apnea is observed.

FIG. 19 is an illustration showing a positional arrangement of a simplified PSG and various sensors for measuring AHI data with respect to a subject H.

FIG. 20 is a graph showing a histogram of angle-based AHI with respect to a subject C to know a body angle range of the subject C where apnea or low respiration is observed.

FIGS. 21A through 21C are illustrations showing examples of display of data relating to body position.

FIG. 22 is a flowchart showing a flow of overall operations of the sleep evaluation system in FIG. 2.

FIG. 23 is a flowchart showing details on SpO₂ measurement in Step S3 in the flowchart of FIG. 22.

FIG. 24 is a flowchart showing details on body angle detection in Step S3 in the flowchart of FIG. 22.

FIG. 25 is a flowchart showing details on SpO₂ measurement data analysis in Step S7 in the flowchart of FIG. 22.

FIG. 26 is a flowchart showing details on body angle measurement data analysis in Step S7 in the flowchart of FIG. 22.

FIG. 27 is a flowchart showing details on angle-based ODI detection, i.e. step S9 in the flowchart of FIG. 22.

FIG. 28 is a block diagram showing a simplified arrangement of a sleep support system as a modification of the embodiment of the invention.

FIG. 29 is a graph showing a relation between change of SpO₂ with time, and change of body position with time.

FIG. 30 is an illustration showing how the body position is detected in a conventional arrangement.

FIG. 31 is a top plan view of a pulse oximeter provided with a direction guide.

### DETAILED DESCRIPTION OF THE PREFERRED EMBODIMENTS OF THE INVENTION

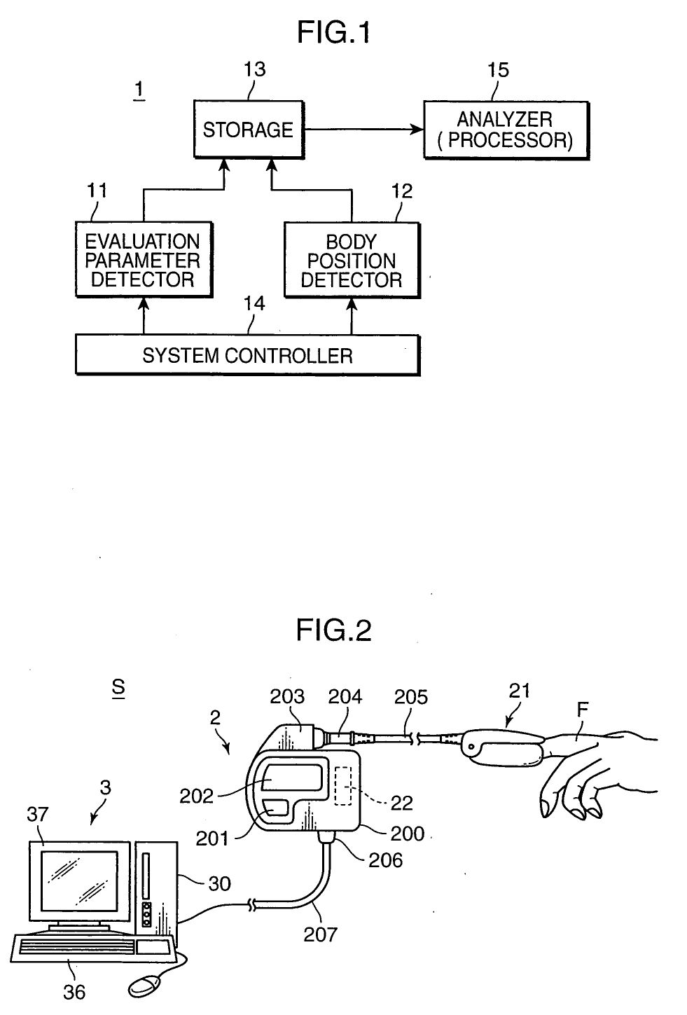

A preferred embodiment of the invention will be described referring to the drawings. First of all, a hardware construction of the embodiment is described. Referring to FIG. 1 schematically showing an entire arrangement of a sleep evaluation system 1 in accordance with the embodiment, the sleep evaluation system 1 is a system for evaluating a correlation between apnea based on a variation in an evaluation parameter having relevancy to apnea, and body tilt angle of a subject in sleep by measuring a body tilt angle of the subject, and the evaluation parameter simultaneously or concurrently at a predetermined sampling frequency, and by expressing the acquired measurement data along a time axis. The sleep evaluation system 1 includes an evaluation parameter detector 11, a body position detector 12, a storage 13, a system controller 14, and an analyzer 15 serving as a processor. Hereinafter, the body tilt angle of the subject is simply called as “body angle”.

The evaluation parameter detector 11 measures a predetermined evaluation parameter of a subject, which is varied due to sleep apnea of the subject. Various parameters which are expressed internally and externally with respect to the subject in association with apnea are adoptable as the evaluation parameter to be measured. In the embodiment, blood oxygen saturation, electroencephalogram, airflow by mouth/nasal breathing, snoring sounds, and movements of chest/abdomen, which are regarded as typical evaluation parameters to be measured in a general PSG, are adoptable. Among these evaluation parameters, it is desirable to use blood oxygen saturation as the evaluation parameter because the blood oxygen saturation can be easily measured with use of a commercially available pulse oximeter or a like device.

The body position detector 12 detects a body position of the subject in terms of angle information. Various angle sensors which are detachably attached to the body trunk portion of the subject or any other appropriate site of the subject and are capable of detecting a body angle of the subject can be used as the body position detector 12. It is preferable to use an angle sensor having a resolution capable of detecting the body angle in the unit of about 5 degrees or less, and particularly preferable to use an angle sensor having a resolution capable of detecting the body angle in the unit of about 1 degree or less. A preferred example of the body position detector 12 is a three-axis acceleration sensor, which will be described later in detail.

The storage 13 stores measurement data obtained by the evaluation parameter detector 11 and the body position detector 12. Examples of the storage 13 include a random access memory (RAM), and an erasable and programmable read only memory (EPROM).

The system controller 14 includes a microprocessor, and controls the evaluation parameter detector 11 and the body position detector 12 to measure the evaluation parameter and the body angle of the subject respectively at a predetermined sampling frequency so that the measurement data obtained by the evaluation parameter detector 11 and the body position detector 12 are stored in the storage 13.

The analyzer 15 analyzes a relation between a variation in the evaluation parameter e.g. a blood oxygen saturation, and the detected body angle of the subject based on the measurement data stored in the storage 13 to obtain a correlation between apnea and body angle of the subject. For instance, the analyzer 15 analyzes ODI, which is an index for judging the degree of severity of SAS, and is acquired with respect to each of the body angles for individual subjects. In the case where measurement data concerning airflow of the respiratory system such as airflow by mouth/nasal breathing and snoring sounds, and movements of the body trunk portion such as movements of chest/abdomen are obtainable as the evaluation parameter in addition to the blood oxygen saturation, it is possible to analyze a correlation between apnea or low respiration, and body angle of the subject. In the latter case, it is desirable to conduct an analysis so that AHI, which is another index for judging the degree of severity of SAS, is obtained with respect to each of the body angles for individual subjects.

The hardware construction of the sleep evaluation system 1 may be arbitrarily designed. The following are some of the examples of the hardware construction of the sleep evaluation system 1.

(a) Solely the sensing devices i.e. the evaluation parameter detector 11 and the body position detector 12 are detachably attached to the subject. A personal computer (hereinafter, called as “PC”) is constituted of the storage 13, the system controller 14, and the analyzer 15. The PC and the sensing devices are connected by a communication line.

(b) A pulse oximeter, which serves as the evaluation parameter detector 11 and is adapted to measure the blood oxygen saturation, is equipped with the body position detector 12, the storage 13, and the system controller 14. The pulse oximeter equipped with these devices is connected to a PC i.e. the analyzer 15 serving as the processor by a USB cable or a like device.

(c) A one-piece system is constructed by additionally providing a function of the analyzer 15 serving as the processor to the pulse oximeter having the arrangement (b).

FIG. 2 is an illustration showing an example of a sleep evaluation system S having the hardware construction (b). The sleep evaluation system S includes a pulse oximeter 2 for concurrently or simultaneously measuring a blood oxygen saturation and a body angle of a subject for storing the measurement data, a PC 3 for reading out the measurement data concerning the blood oxygen saturation and the body angle from the pulse oximeter 2 to analyze a correlation between apnea and body angle of the subject, and a USB cable 207 for connecting the pulse oximeter 2 and the PC 3 for communication according to needs.

The pulse oximeter 2 has a main body 200 and a probe 21. The oximeter main body 200 and the probe 21 are electrically connected by a probe cable 205 equipped with a connector 204. The oximeter main body 200 is externally provided with a power switch 201, an oximeter display 202 with a liquid crystal display, a connecting portion 203 for connecting the oximeter main body 200 to the probe cable 205, and a connecting portion 206 for connecting the oximeter main body 200 to the USB cable 207. The oximeter main body 200 internally has a memory serving as the storage 13, a microprocessor i.e. a central processing unit (CPU) serving as the system controller 14, a power battery, and a three-axis acceleration sensor 22 serving as the body position detector 12. The memory, the microprocessor, and the power battery are not shown in FIG. 2.

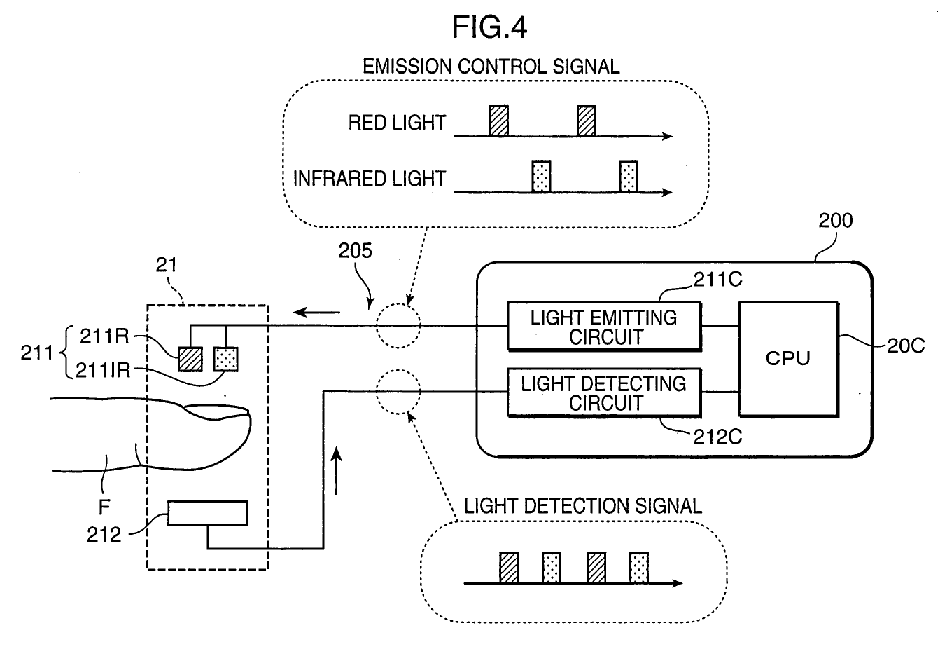

The probe 21 has a paper-clip like shape capable of securely holding a finger F of a subject to measure the blood oxygen saturation of the subject. Specifically, the probe 21 has a pair of holding pieces which are openably jointed to each other so that the probe 21 can securely hold the finger F with a biasing force of a spring or a like member. As will be described later, a light emitter 211 is provided on one of the holding pieces, and a light detector 212 is provided on the other thereof (see FIG. 4).

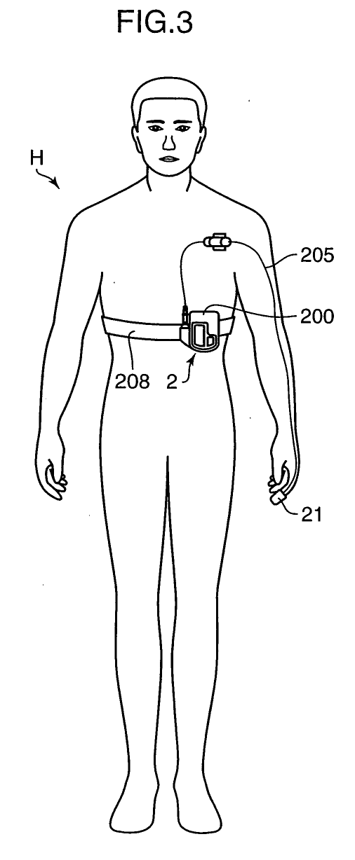

The oximeter main body 200 and the probe 21 are detachably attached to a subject H in the manner as shown in FIG. 3, for instance, in measurement. Specifically, a body belt 208 serving as a fastening device is wound around the body trunk portion of the subject H so that the oximeter main body 200 is fixed to the body trunk portion of the subject H with use of the body belt 208. Also, the probe 21 is fixedly attached to the finger F of the subject H for measurement. Thereafter, the oximeter main body 200 and the probe 21 are connected to each other by way of the probe cable 205. At the time of measurement i.e. during sleep of the subject H, the USB cable 207 is not connected to the oximeter main body 200. The USB cable 207 is connected to the PC 3 after the measurement is completed to read out the measurement data from the pulse oximeter 2.

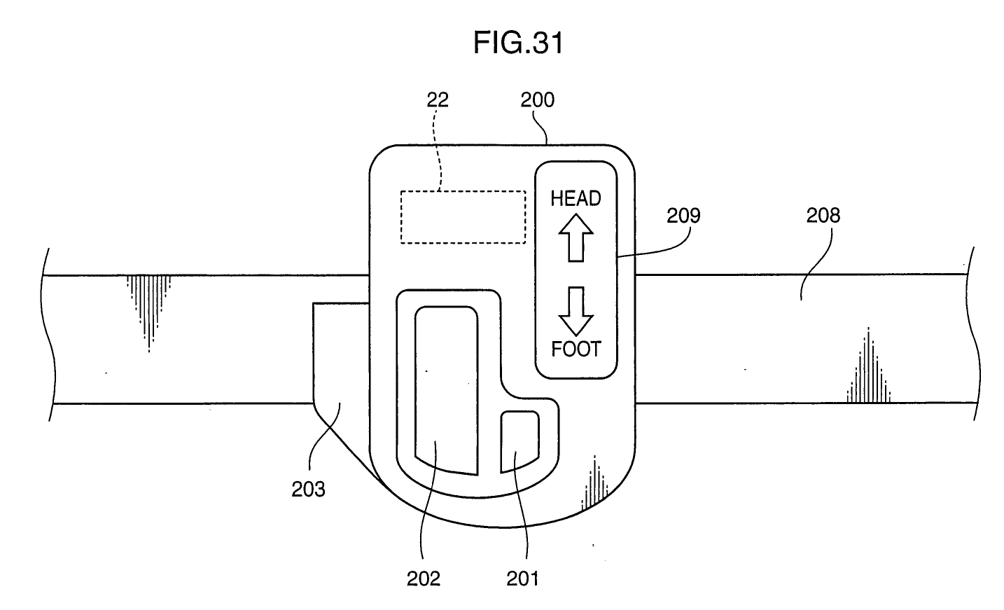

As shown in FIG. 31, it is desirable to provide a direction guide 209 on an outer surface of a casing of the oximeter main body 200 to display a direction in which the pulse oximeter 2 should normally be attached to the subject H so that outputs from the three-axis acceleration sensor 22 with respect to respective axes thereof are accurately obtained as designed in view of a fact that the three-axis acceleration sensor 22 is built in the pulse oximeter 2. Specifically, if the pulse oximeter 2 is inadvertently attached to the subject's body in a direction different from the direction corresponding to the designed axial output of the three-axis acceleration sensor 22, for instance, if the pulse oximeter 2 is attached upside down, plus and minus of X-axis and Y-axis outputs are inverted with respect to the body position of the subject H. In this case, for instance, if the subject rolls over in a rightward direction, such a body position change is misjudged as rolling over in a leftward direction. In view of this, as shown in FIG. 31, the direction guide 209 is provided on the surface of the casing of the oximeter main body 200, wherein “HEAD” and “FOOT” are indicated with arrows to clearly notify a user including the subject H and a medical staff of the direction in which the pulse oximeter 22 should normally be attached to the subject H. This enables to prevent erroneous measurement as described above.

The PC 3 includes a PC main body 30 i.e. a hard disk device, serving as the analyzer 15, an operating unit 36 having a keyboard and the like, and a display unit 37 having a cathode ray tube (CRT) display or a liquid crystal display.

FIG. 4 is an illustration schematically showing a circuit configuration of the probe 21 and the oximeter main body 200 connected thereto. The probe 21 includes the light emitter 211 and the light detector 212. The light emitter 211 has semiconductor light emitting devices for emitting light of two different wavelengths k1, k2, respectively. For instance, one of the semiconductor light emitting devices is a red LED 211R for emitting red LED light of the wavelength k1 in a red wavelength range, and the other one thereof is an infrared LED 211IR for emitting infrared LED light of the wavelength k2 in an infrared wavelength range. The light detector 212 has a photoelectric conversion device for generating an electric current in accordance with an intensity of light emitted from the light emitter 211. An example of the photoelectric conversion device is a silicon photo diode having photosensitivity to at least the wavelengths k1 and k2.

As shown in FIG. 4, the light emitter 211 and the light detector 212 are juxtaposed with respect to the finger F for measurement i.e. living tissue from which the blood oxygen saturation is to be measured. For instance, on the tip of the finger F where a pulse beat of the arterial blood is easily detected optically, the light emitter 211 is arranged adjacent the nail portion of the finger tip, and the light detector 212 is arranged adjacent the ball portion of the finger tip. In an actual measurement, fixedly holding the finger F by the probe 21 enables to dispose the light emitter 211 and the light detector 212 at the aforementioned positions. Alternatively, a medicated tape such as a surgical tape or a first-aid adhesive tape may be used to securely position the light emitter 211 and the light detector 212 with respect to the finger F. By the above attachment, the light of the wavelengths λ1, λ2 which has passed through the finger F is detected by the light detector 212.

The light emitter 211 and the light detector 212 are respectively connected to a light emitting circuit 211C and a light detecting circuit 212C. The light emitting circuit 211C and the light detecting circuit 212C are fabricated in the oximeter main body 200. The light emitter 211 and the light detector 212 are electrically connected to the light emitting circuit 211C and the light detecting circuit 212C, respectively, by way of the probe cable 205.

An operation of the light emitting circuit 211C is controlled by a microprocessor 20C so that a specified emission control signal is issued to the red LED 211R and to the infrared LED 211IR of the light emitter 211. When the emission control signal is issued to the red LED 211R and to the infrared LED 211IR, for instance, the red LED 211R and the infrared LED 211IR are alternately driven, and red light and infrared light are alternately emitted. Also, the light detecting circuit 212C is controlled in synchronism with the emission of the light emitter 211 by the microprocessor 20C to generate an electric current signal i.e. a pulse signal, which is obtained by photoelectrical conversion of the received light in accordance with the received light intensity.

Oxygen is transported by oxidation/reduction of hemoglobin in the blood. The hemoglobin has such optical characteristics that absorption of red light is decreased, and absorption of infrared light is increased when the hemoglobin is oxidized, and, conversely, absorption of red light is increased and absorption of infrared light is decreased when the hemoglobin is reduced. It is possible to obtain the blood oxygen saturation i.e. an arterial blood oxygen saturation by measuring a variation in transmitted light amounts of the red light and the infrared light, which is detected by the light detecting circuit 212C, by utilizing the optical characteristics.

(Description on Three-axis Acceleration Sensor for Detecting Body Angle)

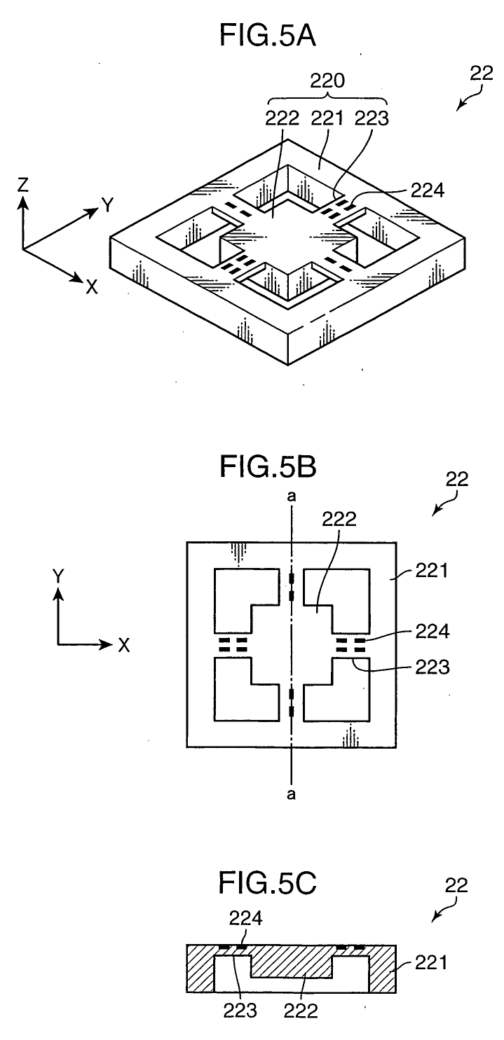

In this section, the three-axis acceleration sensor 22 built in the oximeter main body 200 is described. FIGS. 5A through 5C are illustrations showing a three-axis acceleration sensor using a piezoresistor, as an example of the three-axis acceleration sensor. FIG. 5A is a perspective view of the three-axis acceleration sensor, FIG. 5B is a top plan view thereof, and FIG. 5C is a cross-sectional view taken along the line a-a in FIG. 5B. The three-axis acceleration sensor 22 is constructed utilizing a piesoresistive effect. The piezoresistive effect is such that when a mechanical external force is exerted to an object composed of a semiconductor material having a piezo effect, crystal lattice distortion occurs in the object, and the number of carriers or carrier moving degree in the object is varied, which causes a change in resistance of the object.

The three-axis acceleration sensor 22 includes a sensor body 220 and twelve piezoresistive devices 224. The sensor body 220 has a four-sided frame-like support 221 formed by dry-etching a base material such as silicon, a weight portion 222 disposed in the middle of the support 221, and thin beam portions 223 each for connecting a corresponding side portion of the support 221 to the weight portion 222. The twelve piezoresistive devices 224 are attached to the beam portions 223, as shown in FIG. 5A, for instance. When the weight portion 222 is vibrated by application of acceleration, the beam portions 223 are deformed, and a stress is applied to the piezoresistive devices 224.

Specifically, when an external force is exerted to the three-axis acceleration sensor 22, a tilting force is exerted on the oximeter main body 200. As a result, the weight portion 222 is deformed about X-axis, Y-axis, or Z-axis (see FIG. 5A) depending on the tilting direction of the oximeter main body 200, thereby deforming the beam portions 223. Then, a stress is applied to the piezoresistive devices 224 depending on the degree of the deformation of the beam portions 223, and, as a result, the resistances of the piezoresistive devices 224 are varied depending on the application of the stress. Thus, a tilt angle of the oximeter main body 200 i.e. the body angle of the subject is detected by detecting variations in resistance of the piezoresistive devices 224, which are signals proportional to acceleration.

The acceleration-proportional signals regarding the piezoresistive devices 224 can be detected by constituting a Wheatstone bridge circuit of four piezoresistive devices 224 each for the X-axis, Y-axis, and Z-axis, namely, using the twelve piezoresistive devices 224 in total, and by detecting respective variations in resistance resulting from application of stress to the piezoelectric devices 224 as a voltage change.

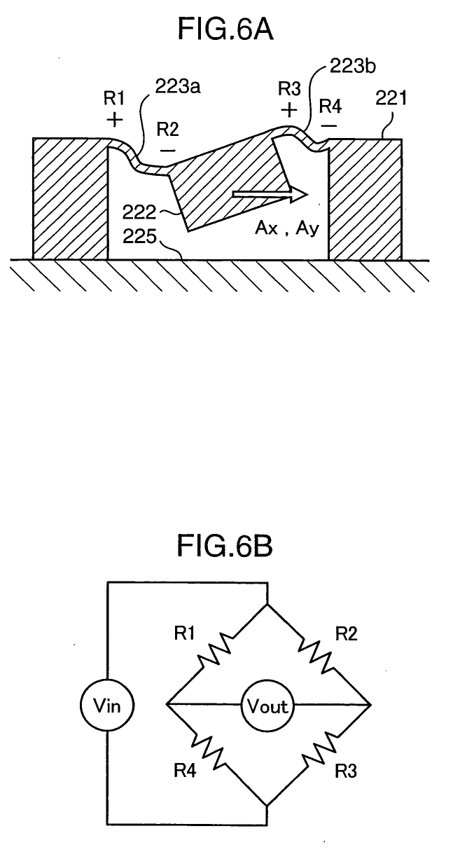

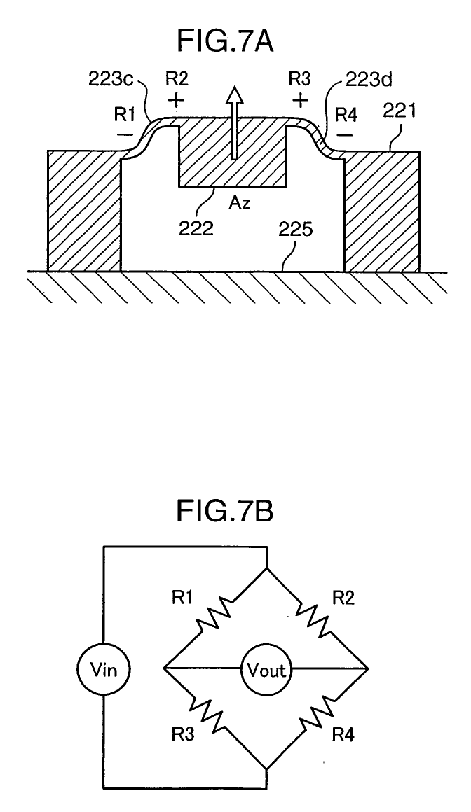

FIG. 6A is an illustration schematically showing deformation of the beam portions 223 i.e. beam portions 223a, 223b in X-axis direction and Y-axis direction, i.e., rotational deformation of the beam portions 223 about the X-axis and the Y-axis. FIG. 6B is a circuit diagram schematically showing a bridge circuit for detecting a voltage change corresponding to the deformation. In FIGS. 6A, 6B, and FIGS. 7A and 7B, which will be described later, symbols R1, R2, R3, and R4 represent four piezoresistive devices 224 in association with one of the X-, Y-, and Z-axes, respectively.

As shown by a deformed beam model in FIG. 6A, when acceleration is applied to the acceleration sensor 22 in the X-axis direction and in the Y-axis direction, a tensile stress is applied to the outer piezoresistive device R1 on the beam portion 223a, with the result that the resistance of the piezoresistive device R1 is increased, and a compressive stress is applied to the inner piezoresistive device R2 on the beam portion 223a, with the result that the resistance of the piezoresistive device R2 is decreased. On the other hand, a tensile stress is applied to the inner piezoresistive device R3 on the beam portion 223b, with the result that the resistance of the piezoresistive device R3 is increased, and a compressive stress is applied to the outer piezoresistive device R4 on the beam portion 223b, with the result that the resistance of the piezoresistive device R4 is decreased. In other words, counteractive resistance variations occur between the piezoresistive devices R1 and R2, and between the piezoresistive devices R3 and R4. Accordingly, in the case where a bridge circuit as shown in FIG. 6B is fabricated, and a constant voltage Vin is applied to the bridge circuit with respect to the X-axis or the Y-axis, an output voltage Vout can be obtained by implementing the equation (1).
Vout={R4/(R1+R4)−R3/(R2+R3)}Vin (1)

FIG. 7A is an illustration schematically showing deformation of the beam portions 223 or beam portions 223c, 223d in Z-axis direction, i.e., vertical deformation of the beam portions 223 in the Z-axis. FIG. 7B is a circuit diagram schematically showing a bridge circuit for detecting a voltage change corresponding to the deformation. The weight portion 222 deforms vertically in response to receiving acceleration in the Z-axis direction. For instance, as shown by a deformed beam model in FIG. 7A, in the case where the weight portion 222 is deformed upwardly, a compressive stress is applied to the outer piezoresistive device R1 on the beam portion 223c, with the result that the resistance of the piezoresistive device R1 is decreased, and a tensile stress is applied to the inner piezoresistive device R2 on the beam portion 223c, with the result that the resistance of the piezoresistive device R2 is increased. On the other hand, a tensile stress is applied to the inner piezoresistive device R3 on the beam portion 223d, with the result that the resistance of the piezoresistive device R3 is increased, and a compressive stress is applied to the outer piezoresistive device R4 on the beam portion 223d, with the result that the resistance of the piezoresistive device R4 is decreased. In other words, counteractive resistance variations occur between the piezoresistive devices R1 and R2, and between the piezoresistive devices R3 and R4. Accordingly, in the case where a bridge circuit as shown in FIG. 7B is fabricated, and a constant voltage Vin is applied to the bridge circuit with respect to the Z-axis, an output voltage Vout can be obtained by implementing the equation (2).
Vout={R3/(R1+R3)−R4/(R2+R4)}Vin (2)

The above describes a basic operation principle as to how the acceleration applied to the oximeter main body 200 is detected by the three-axis acceleration sensor 22.

Next, a principle is described as to how a tilt angle of the oximeter main body 200 is detected with use of the three-axis acceleration sensor 22. The acceleration sensor 22 is an inertial sensor for measuring a velocity component in input axis direction or sensing axis direction i.e. the X-axis direction, the Y-axis direction, and the Z-axis direction shown in FIG. 5A, wherein the velocity component is obtained by subtracting a gravitational acceleration g from a moment acceleration m. Specifically, the velocity component i.e. acceleration A detected by the acceleration sensor 22 is expressed by the equation (3).
A=m−g (3)
Here, let it be assumed that the acceleration sensor 22 is stationary on the ground i.e. m=0, and the gravitational acceleration g along a vertical axis is 1 g. Then, in the case where the direction of the sensing axis coincides with the upwardly extending direction of the vertical axis, the gravitational acceleration g is +1 g, and in the case where the sensing axis is tilted by angle θ with respect to the vertical axis, the gravitational acceleration g equals +1 g multiplied by cos θ.

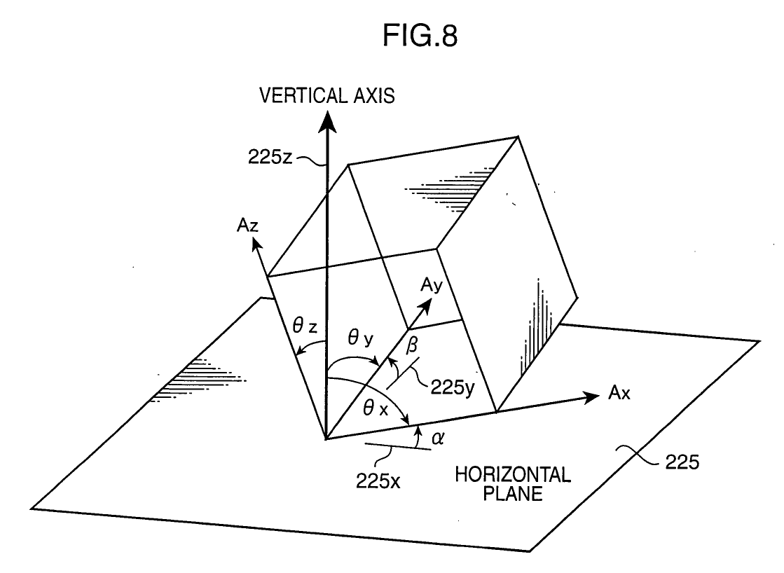

Utilizing the above idea, angles of the X-axis, the Y-axis, and the Z-axis of the acceleration sensor 22 with respect to the vertical axis can be obtained based on gravitational accelerations with respect to the three axes, i.e., the X-, Y-, and Z-axes of the acceleration sensor 22. FIG. 8 is an illustration for defining angles of the X-axis, Y-axis, and Z-axis with respect to the vertical axis in expressing the position of the acceleration sensor 22. Generally, it is proper to express the position of the sensor in terms of angle of the respective axes with respect to the vertical axis. However, in the case where the sensor is in a normal position where the Z-axis coincides with the vertical axis 225z, it is practical to use an angle a defined by the X-axis Ax and a reference line 225x on an imaginary horizontal plane 225, and an angle β defined by the Y-axis Ay and a reference line 225y on the horizontal plane 225, in place of using an angle θx defined by the X-axis Ax and the vertical axis 225z, and an angle θy defined by the Y-axis Ay and the vertical axis 225z, as shown in FIG. 8, to express a tilt of the sensor relative to the normal position. The upward direction on the horizontal plane 225 in FIG. 8 is positive. In view of this, the angles α, β are used to define the tilt of the X-axis Ax and the tilt of the Y-axis Ay with respect to the horizontal plane 225, and the angle θz is used to define the tilt of the Z-axis Az with respect to the vertical axis 225z to express the tilt of the acceleration sensor 22. Using this definition, when the acceleration sensor 22 is not tilted, i.e. the Z-axis Az coincides with the vertical axis 225z, the angles α, β, and θz are all zero, namely, 0 g is outputted from the acceleration sensor 22 with respect to the X-axis, Y-axis, and Z-axis.

Specifically, output values Vx, Vy, and Vz with respect to the X-axis, Y-axis, and Z-axis are obtained by implementing the equations (4), (5), and (6) with use of the angles α, β, and θz, respectively.
Vx=X₀+Xₛ·sin α (4)
Vy=Y₀+Yₛ·sin β (5)
Vz=Z₀+Zₛ·cos θz (6)
where X₀, Y₀, and Z₀ are correction amounts to be added in the respective equations (4), (5), and (6) to cancel initial displacement of the acceleration sensor 22 with respect to the vertical axis. These correction amounts are added to correct an error resulting from positional displacement of the Z-axis of the acceleration sensor 22 with respect to the vertical axis of the oximeter main body 200. Also, Xₛ, Yₛ, and Zₛ represent sensitivities of the acceleration sensor 22 with respect to the X-, Y-, and Z-axes, i.e., count values of outputs from the acceleration sensor 22 with respect to the X-, Y-, and Z-axes per 1 g, which are constants, respectively.

A relation is defined as expressed by the equation (7) regarding tilt angles of the three axes with respect to the vertical axis. Obtaining two of the tilt angles in the equation (7) enables to obtain the remaining one of the tilt angles.
sin²α+sin²β+cos²θz=1 (7)

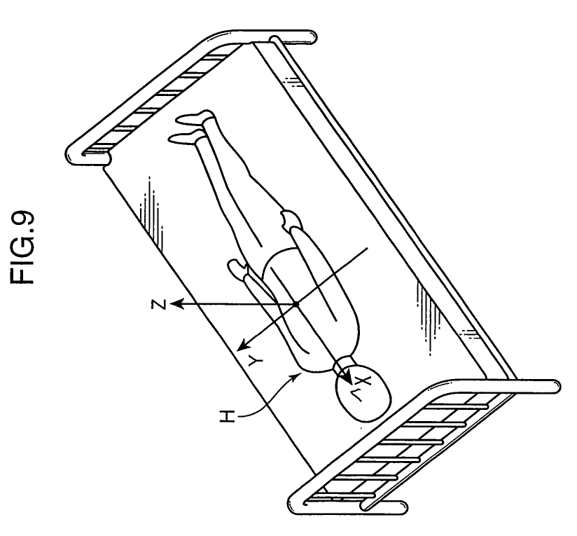

The three-axis acceleration sensor 22 may be built in the oximeter main body 200 so that the respective axes of the acceleration sensor 22 coincide with X-, Y-, and Z-axes shown in FIG. 9 for instance in association with a lying position of the subject. Specifically, in the case where the oximeter main body 200 is attached to the body trunk portion of the subject H in a supine position in the manner as shown in FIG. 3, the acceleration sensor 22 is built in the oximeter main body 200, with the X-axis corresponding to a second axis of the acceleration sensor 22 extending in a longitudinal direction of the subject's body, the Y-axis corresponding to a first axis of the acceleration sensor 22 extending in a sideways direction of the subject's body, and the Z-axis corresponding to a third axis of the acceleration sensor 22 extending in a depthwise direction of the subject's body.

In the above state, when the subject H makes a movement around the Y-axis, the acceleration sensor 22 detects whether the subject H is in a seated position, in other words, whether the subject H is in a standing position or in a lying position, based on a tilt angle of the X-axis i.e. an output value from the acceleration sensor 22 with respect to the X-axis. Also, in the case where the subject H rolls over around the X-axis, the acceleration sensor 22 detects a body angle of the subject H i.e. the position of the subject H based on a tilt angle of the Y-axis i.e. an output value from the acceleration sensor 22 with respect to the Y-axis. Further, the acceleration sensor 22 detects whether the subject H is in a supine position or a prone position based on the symbol (plus or minus) of the tilt angle of the Z-axis i.e. an output value from the acceleration sensor 22 with respect to the Z-axis.

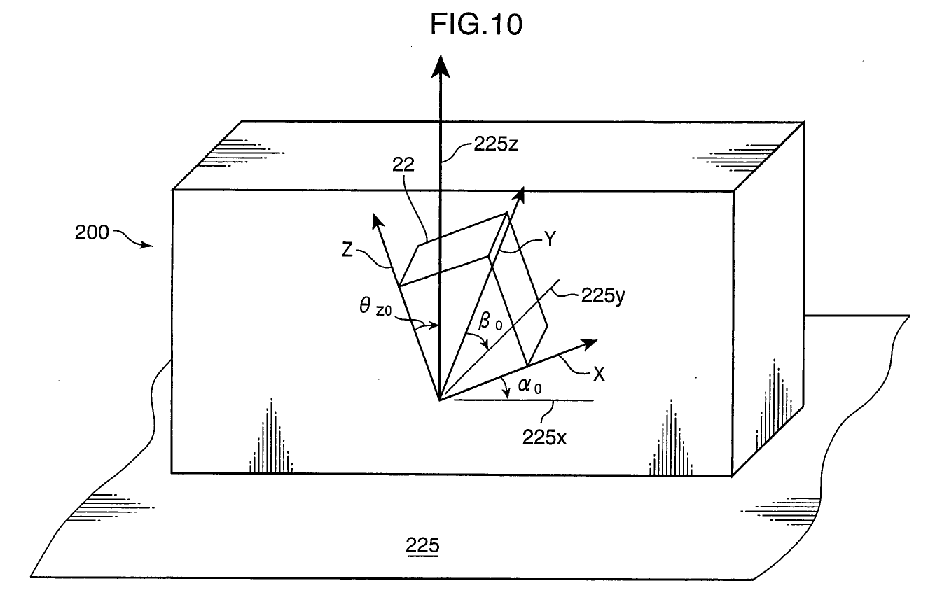

FIG. 10 is an illustration schematically showing a state that the three-axis acceleration sensor 22 is built in the oximeter main body 200. There is no point to be considered if the vertical axis of the oximeter main body 200 i.e. the Z-axis in FIG. 9 in which the oximeter main body 200 is attached to the subject H according to the predetermined manner completely coincides with the Z-axis Az of the acceleration sensor 22. Normally, however, it is not always the case that the vertical axis of the oximeter main body 200 and the Z-axis Az of the acceleration sensor 22 completely coincide with each other, and there remains a tilt of the Z-axis Az with respect to the vertical axis due to the attachment error. Accordingly, the X-axis Ax and the Y-axis Ay of the acceleration sensor 22 are also tilted with respect to the horizontal plane. Specifically, as shown in FIG. 10, the Z-axis is tilted by an angle θz₀ with respect to the vertical axis 225z due to the attachment error, and the X-axis and the Y-axis are tilted by angles α₀ and β₀ with respect to the reference lines 225x and 225y on the horizontal plane 225, respectively. Accordingly, it is required to correct such initial tilts.

Correction amounts to cancel the initial tilts can be obtained by making output values from the acceleration sensor 22 with respect to the X-, Y-, and Z-axes coincident with 0 g when the oximeter main body 200 is placed still on the horizontal plane. Specifically, initial output values Vx₀, Vy₀, and Vz₀ from the acceleration sensor 22 with respect to the X-, Y-, and Z-axes when the oximeter main body 200 is placed still on the horizontal plane are expressed by the equations (8), (9), and (10), which are derived from the equations (4), (5), and (6), respectively.
Vx₀=X₀+Xₛ·sin α₀ (8)
Vy₀=Y₀+Yₛ·sin β₀ (9)
Vz₀=Z₀+Zₛ·cosθz₀ (10)

The output 0 g indicates a state that the Z-axis of the acceleration sensor 22 is not tilted with respect to the vertical axis of the oximeter main body 200. In other words, this state corresponds to α₀=0, β₀=0, and θz₀=0. Substituting these equations in the equations (8), (9), and (10) and implementing the equations (11), (12), and (13) enables to obtain the correction amounts X₀, Y₀, and Z₀ to cancel the initial tilts.
X₀=Vx₀ (11)
Y₀=Vy₀ (12)
Z₀=Vz₀−Z₈ (13)
The correction amounts X₀, Y₀, and Z₀ are analog-to-digitally converted into digital values, and the digital values are stored in the memory provided in the pulse oximeter 2, as correction amount count values.

Next, description is made as to how the body position of the subject H is detected based on the output values from the acceleration sensor 22 with respect to the X-, Y-, and Z-axes. First, the output value from the acceleration sensor 22 with respect to the X-axis is used to detect whether the subject H is in a seated position. Assuming that Px is a count value of the output from the acceleration sensor 22 with respect to the X-axis after A/D conversion, the count value Px is obtained by implementing the equation (14) based on the equation (4).
Px=Px₀+Pxs·sin α (14)
where Px₀ is a count value of the correction amount X₀ after A/D conversion; and Pxₛ is a count value (constant) of the output from the acceleration sensor 22 with respect to the X-axis per 1 g after AID conversion.

The tilt angle α of the X-axis can be obtained by implementing the equation (15). When α≧45°, it is judged that the subject H is in a seated position, and when α<45°, it is judged that the subject H is in a lying position.

$$α = {sin}^{- 1} [ \frac{{P}_{x} - {P}_{x 0}}{{P}_{xS}} ] \qquad ( 15 )$$

Subsequently, the output value from the acceleration sensor 22 with respect to the Y-axis is used to detect the body angle of the subject H. Assuming that Py is a count value of the output from the acceleration sensor 22 with respect to the Y-axis after A/D conversion, the count value Py is obtained by implementing the equation (16) based on the equation (5).
Py=Py₀+Pys·sin β (16)
where Py₀ is a count value of the correction amount Y₀ after A/D conversion, and Pys is a count value (constant) of the output from the acceleration sensor 22 with respect to the Y-axis per 1 g after A/D conversion.

The tilt angle β of the Y-axis can be obtained by implementing the equation (17). In the equation (17), the angle β is 180° or less.

$$β = {sin}^{- 1} [ \frac{{P}_{y} - {P}_{y 0}}{{P}_{y S}} ] \qquad ( 17 )$$

In implementing the equation (17), two cases satisfy the equation: Py−Py₀=0, namely, a case where β=0°, which corresponds to a supine position, and a case where β=180°, which corresponds to a prone position. The count value of the output from the acceleration sensor 22 with respect to the Z-axis is used to judge whether the computation results represents a supine position or a prone position. Specifically, when β=0°, θz=0°. Accordingly, the count value of the output from the acceleration sensor 22 with respect to the Z-axis is positive i.e. a count value per +1 g. On the other hand, when β=180°, θz=180°. Accordingly, the count value of the output from the acceleration sensor 22 with respect to the Z-axis is negative i.e. a count value per −1 g. This enables to make a judgment as to whether the computation results represents a supine position or a prone position.

The tilt angle of the Z-axis can be also obtained by the following approach. Assuming that Pz is a count value of the output from the acceleration sensor 22 with respect to the Z-axis after A/D conversion, the count value Pz is obtained by implementing the equation (18).
Pz=Pz₀+Pzs·cos θz (18)
where Pz₀ is a count value of the correction amount Z₀ after A/D conversion, and Pzs is a count value (constant) of the output from the acceleration sensor 22 with respect to the Z-axis per 1 g after A/D conversion.

The tilt angle θz of the Z-axis can be obtained by implementing the equation (19).

$$θ z = {cos}^{- 1} [ \frac{{P}_{z} - {P}_{z 0}}{{P}_{z S}} ] \qquad ( 19 )$$

(Description on Electrical Configuration)

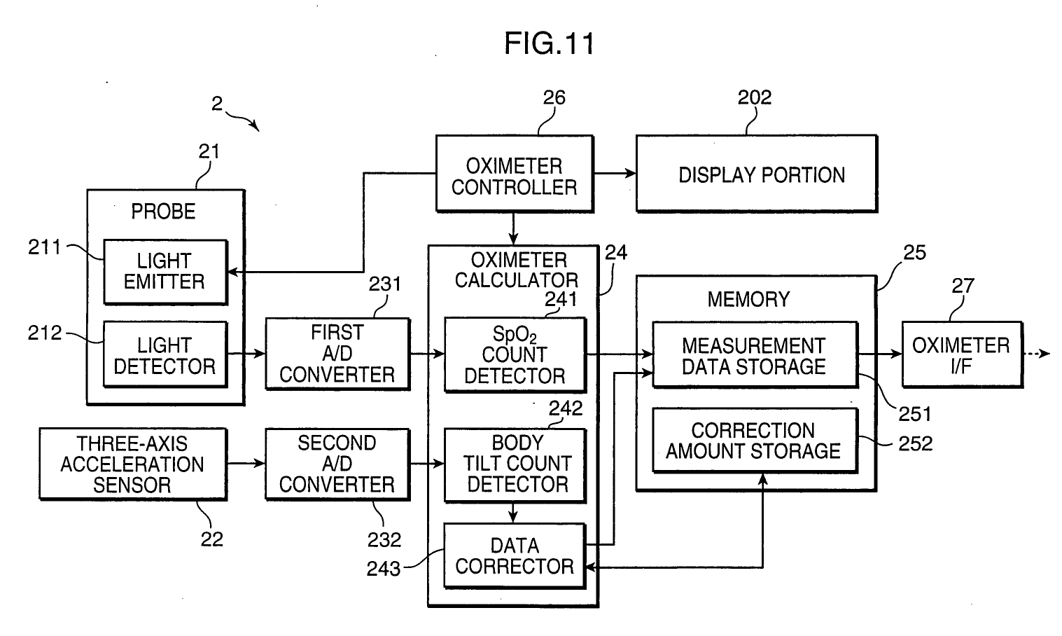

FIG. 11 is a block diagram of an arrangement showing electrical functions of the pulse oximeter 2. The pulse oximeter 2 includes a first A/D converter 231, a second A/D converter 232, an oximeter calculator 24, a memory 25 serving as the memory, an oximeter controller 26, and an oximeter interface (I/F) 27 in addition to the oximeter display 202, the probe 21 serving as a blood oxygen saturation measuring device, and the three-axis acceleration sensor 22 serving as the body tilt detector.

As mentioned above, the probe 21 has the light emitter 211 and the light detector 212 to acquire measurement data concerning the blood oxygen saturation of the subject. Also, the three-axis acceleration sensor 22 acquires measurement data concerning the body angle of the subject.

An analog current signal outputted from the light detector 212 at a predetermined sampling frequency in accordance with the transmitted amounts of red light and infrared light is converted into a voltage signal by a current/voltage converting circuit (not shown), and the voltage signal is converted into a digital signal by the first A/D converter 231. Similarly, respective output values i.e. analog current signals from the three-axis acceleration sensor 22 with respect to the X-, Y-, and Z-axes are converted into voltage signals corresponding to the aforementioned output values Vx, Vy, and Vz, and then these voltage signals are converted into digital signals by the second A/D converter 232.

The oximeter calculator 24 is a functioning part for obtaining count values corresponding to blood oxygen saturation (SpO₂) and body angle based on the digital measurement signals outputted from the first A/D converter 231 and from the second A/D converter 232, respectively. The oximeter calculator 24 includes an SpO₂ count detector 241, a body tilt count detector 242, and a data corrector 243.

The SpO₂ count detector 241 detects a count value corresponding to SpO₂ every predetermined cycle e.g. every one second in response to receiving the digital measurement signal from the first A/D converter 231 at a fixed interval. The body tilt count detector 243 detects count values corresponding to respective tilts of the X-, Y-, and Z-axes i.e. the aforementioned Px, Py, and Pz every predetermined cycle in response to receiving the digital measurement signal from the second A/D converter 232 at a fixed interval.

The data corrector 243 is a functioning part for correcting the count values corresponding to the respective tilts of the X-, Y-, and Z-axes detected by the body tilt count detector 243 by an amount corresponding to axial displacement resulting from the attachment error of the three-axis acceleration sensor 22 to the pulse oximeter 2. Specifically, the data corrector 243 corrects the count values corresponding to the tilts of the X-, Y-, and Z-axes detected by the body tilt count detector 243, using the correction amount count values X₀, Y₀, and Z₀ with respect to the X-, Y-, and Z-axes, which are obtained by implementing the equations (11), (12), and (13), respectively.

The memory 25 includes e.g. a RAM or a like device, and has a measurement data storage 251 and a correction amount storage 252. The measurement data storage 251 temporarily stores the measurement data acquired by the probe 21 and by the three-axis acceleration sensor 22 i.e. the count values corresponding to the respective measurement data in association with the time when the respective data have been acquired. The correction amount storage 252 stores correction amount count values obtained by analog-to-digitally converting the analog correction amounts X₀, Y₀, and Z₀ with respect to the X-, Y-, and Z-axes, which are used in correcting the count values corresponding to the tilts of the X-, Y, and Z-axes by the data corrector 243.

The oximeter controller 26 controls sensing operations by the probe 21 i.e. the light emitter 211 and the light detector 212, and by the three-axis acceleration sensor 22, an operation of calculating the count values by the oximeter calculator 24, and an operation of writing the count values into the memory 25. Specifically, the oximeter controller 26 causes the probe 21 and the three-axis acceleration sensor 22 to acquire the measurement data concerning SpO₂ and body angle of the subject at the predetermined sampling frequency, causes the oximeter calculator 24 to calculate the respective count values corresponding to the measurement data, and causes the memory 25 to store the obtained count values therein.

The oximeter I/F 27 is an interface for connecting the PC 3 and the pulse oximeter 2 for data communication. Specifically, the oximeter I/F 27 functions as an interface for downloading the count values corresponding to the measurement data stored in the memory 25 of the pulse oximeter 2 to the PC 3.

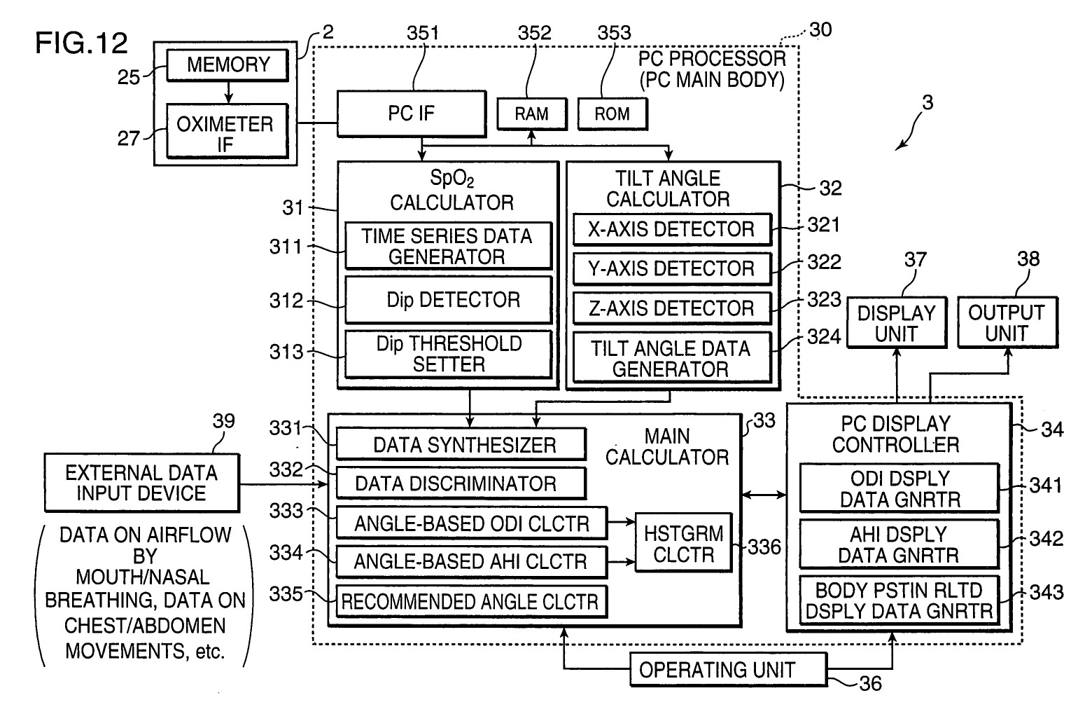

FIG. 12 is a block diagram of an arrangement primarily showing electrical functions of a PC main body 30 i.e. an analyzer or a processor of the PC 3. The PC main body 30 includes an SpO₂ calculator 31, a tilt angle calculator 32, a main calculator 33, a PC display controller 34, a PC interface (I/F) 351, an RAM 352, and an ROM 353.

The SpO₂ calculator 31 is a functioning part for obtaining the number of times when the SpO₂ is lowered due to apnea of the subject, and includes a time series data generator 311, a Dip detector 312, and a Dip threshold setter 313.

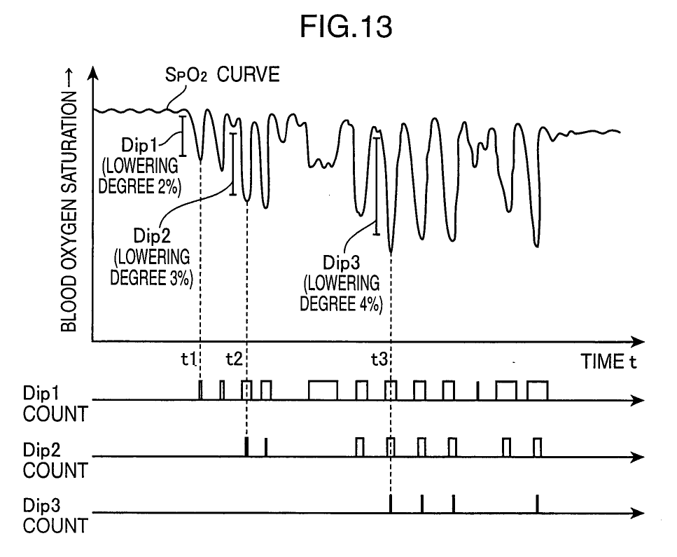

The time series data generator 311 creates data concerning an SpO₂ curve by expressing the count values corresponding to the SpO₂, which have been acquired from the measurement data storage 251 of the memory 25 of the pulse oximeter 2 in association with the data acquired time, along a time axis. FIG. 13 is a graph showing an example of the SpO₂ curve. Expressing the SpO₂ count values acquired at the predetermined sampling frequency along the time axis enables to obtain one SpO₂ curve with respect to the subject. In the case where sleep apnea occurred in the subject, the SpO₂ is lowered. In other words, the SpO₂ curve shows a plurality of peaks where the SpO₂ is temporarily lowered. Hereinafter, a peak where the SpO₂ is lowered is called as “Dip”. For instance, in FIG. 13, the times t1, t2, and t3 correspond to Dips.

The Dip detector 312 detects a Dip having relevancy to apnea of the subject based on the data concerning the SpO₂ curve created by the time series data generator 311. The Dip threshold setter 313 sets a Dip detection threshold in detecting a “significant Dip” by the Dip detector 312.

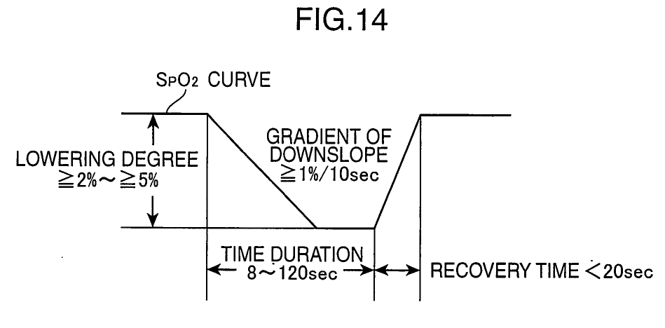

Detecting a Dip corresponds to detecting an event of apnea or low respiration in the measurement data acquired concerning the subject. FIG. 14 is an illustration schematically showing indexes for detecting a Dip. Examples of the index for Dip detection include a gradient of downslope of the SpO₂ curve, a lowering degree of SpO₂, a time duration when lowering of the SpO₂ is continued, and a time required for the subject to recover to his or her normal sleep state, i.e. a recovery rate. In this embodiment, Dip detection by the Dip detector 312 and by the Dip threshold setter 313 can be defined, as shown in FIG. 14 to judge whether a detected Dip of the SpO₂ curve is a significant Dip when the following requirements are satisfied, for instance.
time duration when lowering of SpO₂ is continued: 8 to 120 sec.,
gradient of downslope of SpO₂ curve: >1%/10 sec.,
lowering degree of SpO₂: >2% to >5%, and
time required for recovery: <20 sec.

The Dip in this detection is not obtained based on a lowering degree relative to a. certain reference value, but is obtained based on a lowering degree relative to a certain point of the SpO₂ curve which is varied with time, i.e., a certain point of time during a sleeping time of the subject. For instance, in the case where there are defined three thresholds Dip1, Dip2, and Dip3, e.g., thresholds 2%, 3%, and 4% regarding the lowering degree of Dip, as shown in FIG. 13, Dips that show lowering relative to the start points of time when the respective Dips occurred by the respective thresholds are used as a detection index, in place of Dips that show lowering relative to the initial SpO₂ at the measurement start time by the respective thresholds. This technique enables to accurately detect lowering of the SpO₂.

In the SpO₂ curve illustrated in FIG. 13, if the Dip threshold setter 313 sets the lowering degree of 2% as a threshold for Dip detection, the Dip detector 312 judges that a Dip is found if the lowering degree exceeds the threshold and the other indexes satisfy the aforementioned requirements, and then, the Dip detector 312 counts the event as one Dip. Binary signals on the timeline indicated by “Dip1 COUNT” in FIG. 13 represent the number of Dips which are counted on the basis of lowering degree of 2%. Likewise, binary signals on the timeline indicated by “Dip2 COUNT” represent the number of Dips counted on the basis of lowering degree of 3%, and binary signals on the timeline indicated by “Dip3 COUNT” represent the number of Dips counted on the basis of lowering degree of 4%, respectively. Data concerning the SpO₂ curve, and data concerning the number of Dips obtained in the SpO₂ calculator 31 are sent to the main calculator 33.

The tilt angle calculator 32 is a functioning part for calculating data concerning change of the body angle of the subject with time during the sleeping time of the subject, and includes an X-axis tilt angle detector 321, a Y-axis tilt angle detector 322, a Z-axis tilt angle detector 323, and a tilt angle data generator 324.

The X-axis tilt angle detector 321 obtains data concerning change of the tilt angle a of the X-axis with time based on the equation (15), using the count value Px corresponding to the tilt of the X-axis of the three-axis acceleration sensor 22, which is outputted from the measurement data storage 251 of the memory 25 of the pulse oximeter 2. Likewise, the Y-axis tilt angle detector 322 obtains data concerning change of the tilt angle β of the Y-axis with time based on the equation (17), using the count value Py corresponding to the tilt of the Y-axis of the three-axis acceleration sensor 22, and the Z-axis tilt angle detector 323 obtains data concerning change of the tilt angle θz of the Z-axis with time based on the equation (19), using the count value Pz corresponding to the tilt of the Z-axis of the three-axis acceleration sensor 22.

The tilt angle data generator 324 calculates a time period when the subject is in a seated position, e.g., a time period when the tilt angle α≧45°, based on the time-based change of the tilt angle a of the X-axis detected by the X-axis tilt angle detector 321. Also the tilt angle data generator 324 obtains data concerning a time-based change of the body angle of the subject based on the time-based change of the tilt angle β of the Y-axis detected by the Y-axis tilt angle detector 322, namely, a change of the body position of the subject, and based on the time-based change of the tilt angle θz of the Z-axis detected by the Z-axis tilt angle detector 323, namely, a judgment as to whether the subject is in a supine position or a prone position.

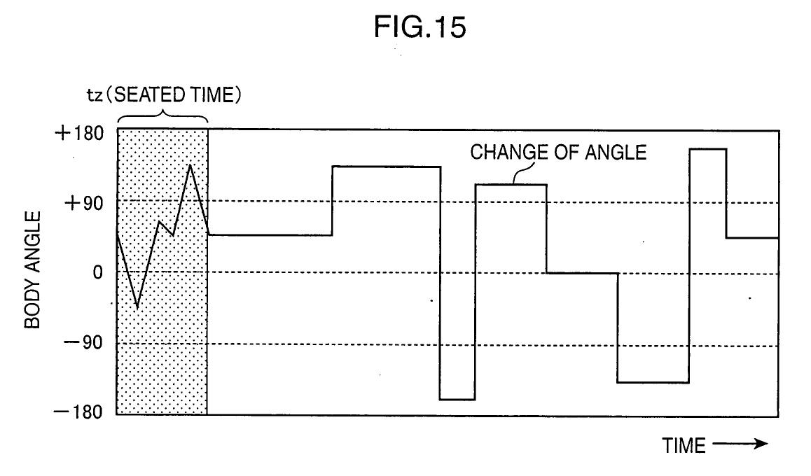

FIG. 15 is a time chart schematically showing an example of data concerning time-based change of the body angle of the subject generated by the tilt angle data generator 324. As shown in FIG. 15, the tilt angle data generator 324 generates data concerning the body position of the subject in terms of angle information represented by “BODY ANGLE”, in place of the roughly classified body directions as in the conventional art. Also, since this arrangement enables to obtain a seated time tz when the subject is in a seated position based on the tilt angle a of the X-axis, the seated time tz in the time chart can be extracted as discriminated data. It is often the case that the tilt angle β of the Y-axis is continuously changed during a seated time including a walking time, unlike a sleeping time. In the example of FIG. 15, the body angle is continuously changed in the seated time tz. The data concerning time-based change of the body angle obtained in the tilt angle calculator 32 is sent to the main calculator 33.

The main calculator 33 is a functioning part for obtaining a relation between apnea or low respiration, and body angle of the subject, and includes a data synthesizer 331, a data discriminator 332, an angle-based ODI calculator 331, an angle-based AHI calculator 334, a recommended angle calculator 335, and a histogram calculator 336.

The data synthesizer 331 creates composite data by expressing the data concerning the SpO₂ curve and the number of Dips outputted from the SpO₂ calculator 31, and the data concerning the time-based change of body angle outputted from the tilt angle calculator 32 along a common time axis. By the data synthesis, basic data for obtaining a correlation between apnea and body angle of the subject can be obtained.

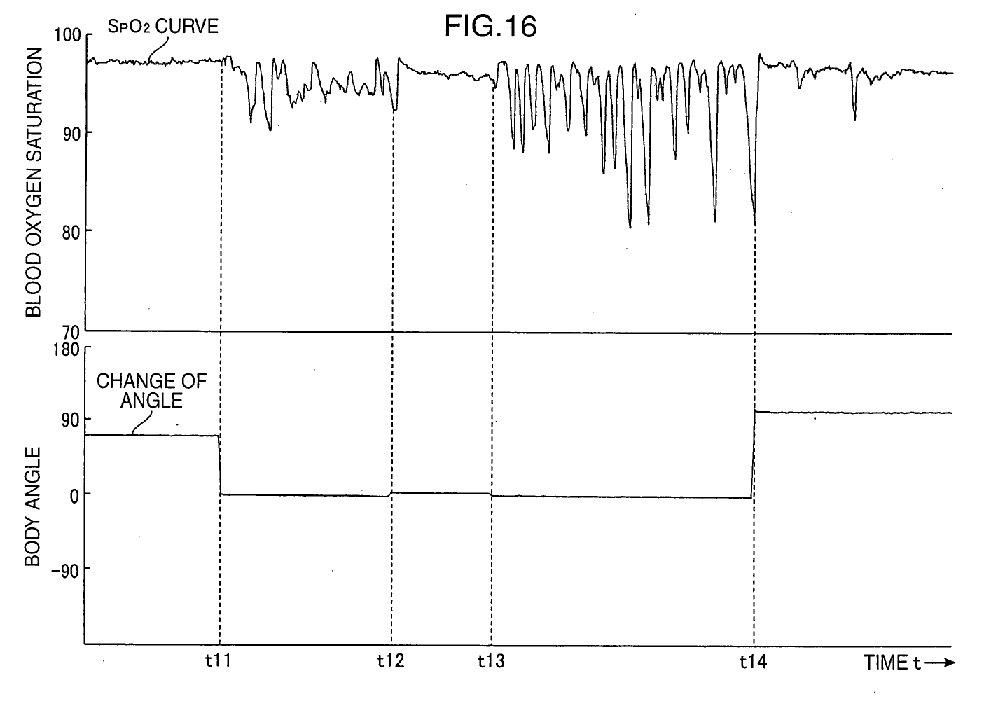

FIG. 16 is a time chart showing an example of the composite data created by the data synthesizer 331 in a graph. As shown in the time chart, a drastic SpO₂ lowering i.e. a Dip is not found until the point of time t11 when the subject is supposed to be in a position close to a lateral decubitus position, and the time after the point of time t14. However, several Dips are found during a time period from t11 to t14 when the subject is supposed to be in a position close to a supine position. Also, observing a relation between change of body angle, and occurrence of Dip concerning the SpO₂ during the time period from t11 to t14, Dip occurs during a time period from t11 to t12, and Dip does not occur after the point of time t12 at which the body angle is slightly changed. Further, at the point of time t13 when the body angle is slightly changed, namely, is returned to a body angle close to the body angle in the time period from t11 to t12, Dip occurs. In this way, a correlation between apnea and body angle of the subject during the time period from t11 to t14 can be accurately obtained by acquiring data concerning the body position of the subject in terms of angle information, whereas, in the conventional arrangement, the body position corresponding to the time period from t11 to t14 is simply judged as a supine position.

The data discriminator 332 discriminates and extracts data corresponding to the time period when analysis on ODI or AHI is to be executed with respect to all the composite data created by the data synthesizer 331. The data discrimination can be performed based on a command signal issued from the operating unit 36. Alternatively, the data discriminator 332 may invalidate data which is attached with an identification code indicating that the data is acquired in the seated time tz (see FIG. 15) by the tilt angle data generator 324, from among all the composite data.

The angle-based ODI calculator 333 screens the composite data created by the data synthesizer 331 with respect to each of the body angles as shown in FIG. 16, for instance, and counts the number of Dips with respect to each of the body angles. The angle-based ODI is an index, which represents the number of Dips counted on the basis of body angle, unlike the conventional ODI, which is counted on the basis of time.

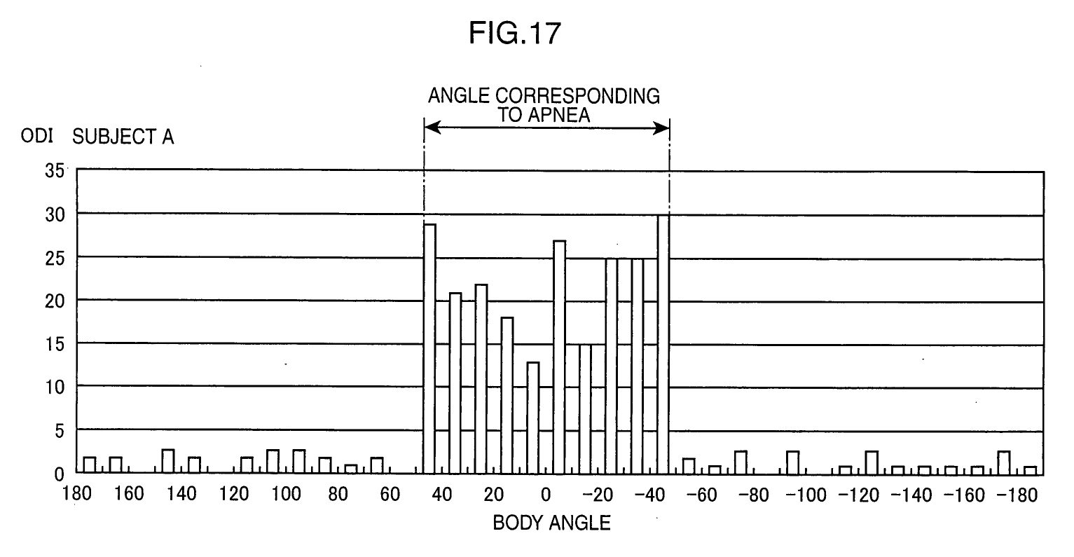

It is possible to create a histogram relating to the Dip number in terms of body angle with respect to a specific subject based on a computation result of the angle-based ODI calculator 333. FIG. 17 is an example of a histogram obtained with respect to a subject A. In case of the subject A, the number of Dip is large when the body angle lies in the range from 40° to −50°. In other words, it is judged that apnea is likely to occur when the body angle is in the aforementioned range. In view of this, it is possible to treat the subject A by suggesting use of a pillow that enables to secure the body angle at 40° or larger so that occurrence of apnea may be suppressed.

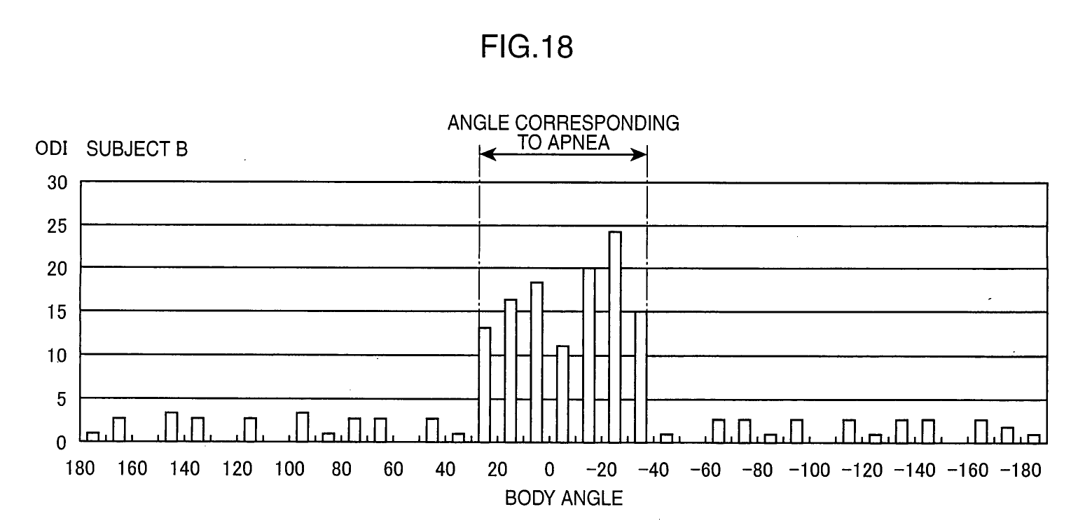

FIG. 18 is an illustration showing an example of a histogram obtained with respect to another subject B. In case of the subject B, the number of Dip is large when the body angle lies in the range from 20° to −40°. Particularly, Dip occurs frequently in a wide range when the body angle is minus. In view of this, it is possible to treat the subject B by suggesting use of a pillow that enables to secure the body angle at 20° or larger. In this way, since the angle-based ODI can be obtained by the angle-based ODI calculator 333, this arrangement enables to provide individual subjects with accurate diagnosis depending on body position.

The angle-based AHI calculator 334 screens AHI data concerning airflow by mouth/nasal breathing, snoring sounds, movements of chest/abdomen, and movements of leg muscles, which is necessary for calculating AHI and is expressed along a time axis, in addition to the composite data concerning SpO₂ and body angle that is created by the data synthesizer 331 with respect to each of the body angles by referring thereto, and counts the number of Dips resulting from apnea or low respiration with respect to each of the body angles. The angle-based AHI is an index, which represents the number of Dips counted on the basis of body angle, unlike the conventional AHI, which is counted on the basis of time. In this arrangement, as shown in FIG. 12, it is possible to input measurement data which has been acquired by a measuring device other than the pulse oximeter 2 to the main controller 33, as the AHI data, by way of an external data input device 39.

Alternatively, it is possible to provide the pulse oximeter 2 with a function of integrally acquiring measurement data necessary for calculating AHI other than the SpO₂ for storage, and to download the measurement data along with the SpO₂ count values. The pulse oximeter provided with this function is called as “simplified PSG” hereinafter.

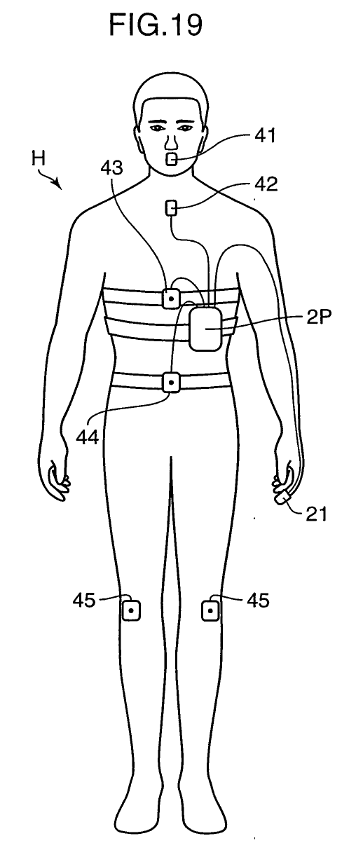

FIG. 19 is an illustration showing a positional arrangement of the simplified PSG 2P, and various sensors for detecting AHI data with respect to a subject H. FIG. 19 shows a positional arrangement concerning sensors of a well-known polysomnograph (PSG). An airflow sensor 41 for detecting airflow by mouth/nasal breathing, a snoring sound sensor 42 for detecting snoring sounds, a chest sensor 43 for detecting movements of a chest, an abdomen sensor 44 for detecting movements of an abdomen, and leg sensors 45 for detecting movements of leg muscles are attached to the subject H, in addition to the probe 21 for detecting SpO₂. The simplified PSG 2P is attached to the body trunk portion of the subject H.

The simplified PSG 2P is internally provided with a memory for storing measurement data from the respective sensors, and a body tilt detector corresponding to the three-axis acceleration sensor 22. Acquiring predetermined measurement data with respect to the subject H with use of the simplified PSG 2P, and allowing the acquired measurement data to be downloaded to the PC main body 30 enables to cause the angle-based AHI calculator 334 to screen the measurement data with respect to each of the body angles and to count the number of Dips resulting from apnea or low respiration with respect to each of the body angles.

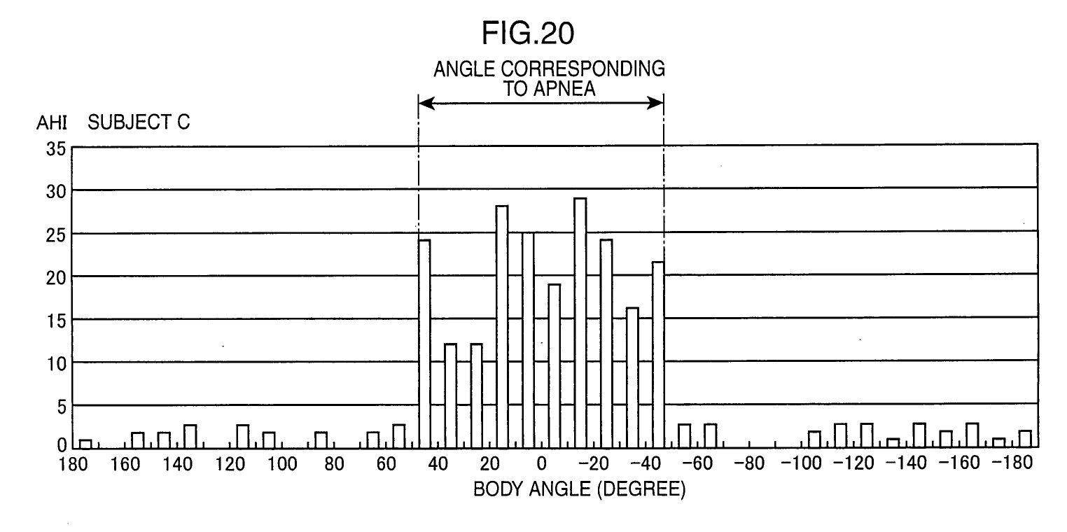

It is possible to create a histogram relating to the Dip number in terms of body angle with respect to a specific subject based on a computation result of the angle-based AHI calculator 334. FIG. 20 is an example of a histogram obtained with respect to a subject C. In case of the subject C, the frequency of occurrence of Dip is large when the body angle lies in the range from 40° to -50°. In other words, it is judged that apnea is likely to occur when the body angle is in the aforementioned range. In view of this, it is possible to treat the subject C by suggesting use of a pillow that enables to secure the body angle at 40° or larger so that occurrence of apnea or low respiration may be suppressed.

The recommended angle calculator 335 calculates a body angle with less or no likelihood of occurrence of Dip in the subject i.e. a body angle having a frequency of occurrence of Dip less than a predetermined number of times based on a computation result of the angle-based ODI calculator 333 or the angle-based AHI calculator 334, and defines the body angle as the recommended body angle with less or no likelihood of apnea. Providing the recommended angle calculator 335 enables to provide the data that readily notifies the subject of the body angle effective in suppressing apnea.

The histogram calculator 336 creates a histogram showing a relation between the body angle and the number of Dips based on the computation result of the angle-based ODI calculator 333 or the angle-based AHI calculator 334. Obtaining the histograms (see FIGS. 17, 18, and 20) enables to allow the subject to grasp the correlation between body angle, and apnea or low respiration.

The PC display controller 34 is a functioning part for displaying the various data calculated in the main calculator 33 on the display unit 37 in the form of a certain image or for outputting the various data to an output unit 38. For instance, the PC display controller 34 generates the composite data image as shown in FIG. 16 created by the data synthesizer 331, and displays the image on the display unit 37. The PC display controller 34 includes an ODI display data generator 341, an AHI display data generator 342, and a body position related display data generator 343.

The ODI display data generator 341 generates predetermined data concerning ODI for display/output in response to receiving display designation from the operating unit 36 by using the data obtained by the angle-based ODI calculator 333 and by the histogram calculator 336. For instance, the ODI display data generator 341 generates the histogram image as shown in FIGS. 17 and 18 for displaying on the display unit 37 or outputting to the output unit 38. Likewise, the AHI display data generator 342 generates predetermined data concerning AHI for display/output in response to receiving display designation from the operating unit 36 by using the data obtained by the angle-based AHI calculator 334 and by the histogram calculator 336. For instance, the AHI display data generator 342 generates the histogram image as shown in FIG. 20 for displaying on the display unit 37 or outputting to the output unit 38. It is desirable to configure the ODI display data generator 341 and the AHI display data generator 342 in such a manner that data for displaying/outputting ODI or AHI in a designated angle range be creatable in response to receiving designation on the body angle range from the operating unit 36

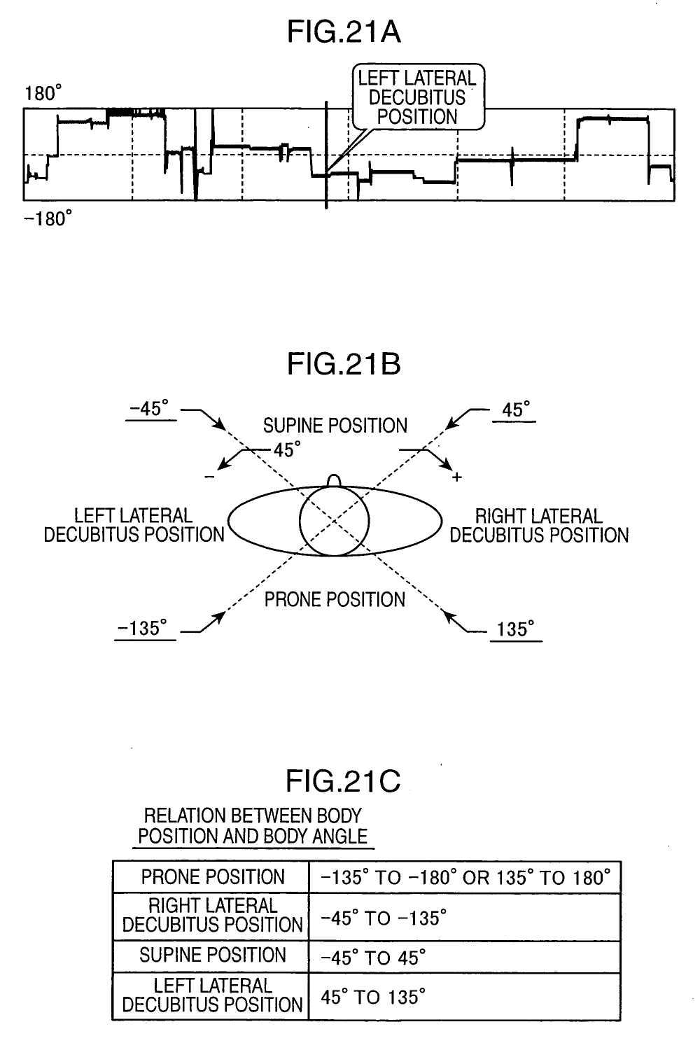

The body position related display data generator 343 converts the body angle data acquired in the tilt angle calculator 32 into a body direction for display. For instance, in the case where data concerning time-based change of body angle, as shown in FIG. 21A is acquired, the body angle data is displayed on the display unit 37, and also display data capable of displaying the body angle data as a body direction is generated in response to designation on an arbitrary point on the graph with use of a cursor. This is performed considering a case that it is convenient to display the body position in terms of body direction rather than angle information that is expressed numerically.

In the above arrangement, as shown in FIG. 21B, for instance, the body position may be classified into four body directions, i.e., a supine position, a prone position, a left lateral decubitus position, and a right lateral decubitus position, and correlations between the respective body directions and body angles may be defined as shown in FIG. 21C. Further alternatively, the four body directions may each be classified into two sub directions, and eight body directions in total may be displayed.

The PC I/F 351 is an interface for connecting the PC main body 30 and the pulse oximeter 2 for data communication. The RAM 352 temporarily stores therein the measurement data downloaded from the memory 25 of the pulse oximeter 2, and various data obtained in the relevant sections of the PC main body 30. The ROM 353 stores therein an operation program for operating the PC main body 30 or the sleep evaluation system S, and the like.

(Description on Operation Flow)

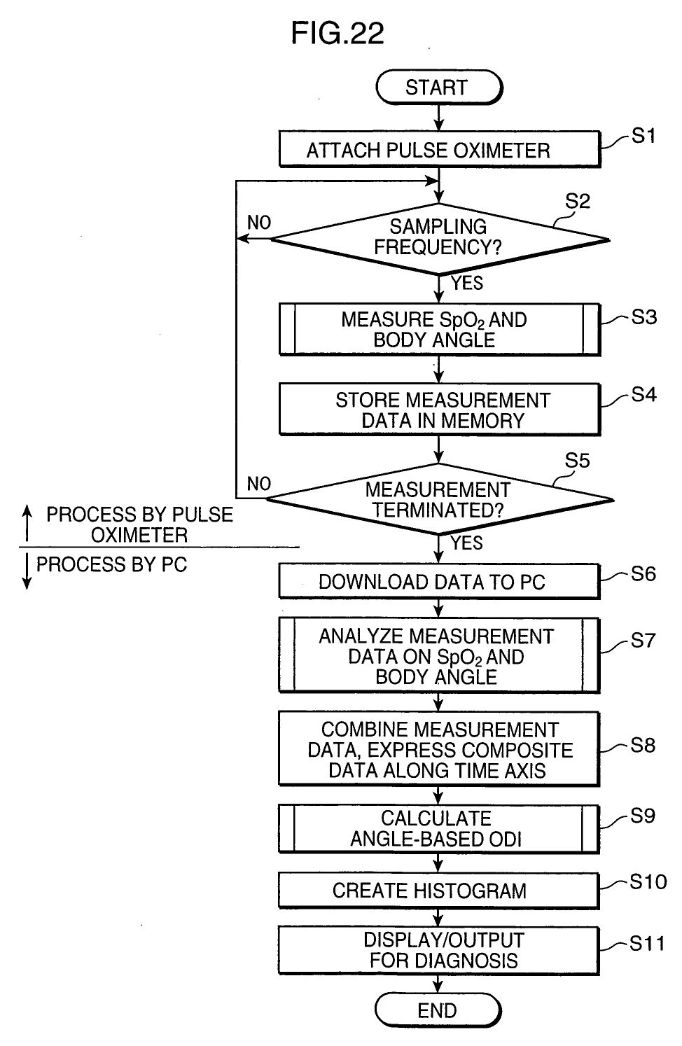

An operation of the sleep evaluation system S having the arrangement is described based on the flowcharts shown in FIGS. 22 through 26, and also referring to the block diagrams of FIGS. 11 and 12 according to needs. FIG. 22 is a flowchart showing a flow of overall operations of the sleep evaluation system S. In this embodiment, described is a flow, in which the pulse oximeter 2 is attached to the subject H, as shown in FIG. 3, the SpO₂ and the body angle are concurrently detected, and the angle-based ODI is obtained with respect to the subject H.

First, the pulse oximeter 2 is attached to the subject's body (Step Si). Specifically, the oximeter main body 200 is attached to the body trunk portion of the subject H with use of the body belt 208 serving as the fastening device, and a finger of the subject H is securely held by the probe 21 (see FIGS. 2 and 3). After completion of these operations, measurement is started. A timer may be set so that a time is started to be measured upon lapse of a certain time, considering a time required for the subject H to fall asleep.

When the measurement is started, it is judged whether the current time is coincident with the time of the predetermined sampling frequency (Step S2). If it is judged that the current time is coincident with the time of the sampling frequency (YES in Step S2), measurement data concerning SpO₂ of the subject H is acquired from the probe 21, and measurement data concerning body angle of the subject H is acquired from the three-axis acceleration sensor 22 (Step S3). Then, after A/D conversion or a predetermined computation is executed, the measurement data is stored in the memory 25 (see FIG. 11) of the pulse oximeter 2 (Step S4).

Then, it is judged whether the measurement is to be terminated (Step S5). In the case where it is judged that the system S is on halfway of the measurement (NO in Step S5), the routine returns to Step S2 to cyclically repeat the operations from Step S2 to Step S4. The measurement is carried on even if the subject wakes up in the middle of sleep such as going to the bathroom. On the other hand, if the predetermined measurement period is ended, or the subject H intentionally terminates the measurement because he or she completely wakes up in the measurement period (YES in Step S5), the measurement operation with use of the pulse oximeter 2 is terminated.

Thereafter, as shown in FIG. 2, the pulse oximeter 2 and the PC 3 are connected by way of the USB cable 207 so that the measurement data stored in the pulse oximeter 2 is downloaded from the pulse oximeter 2 to the PC 3 (Step S6). Specifically, the measurement data concerning SpO₂ and body angle, which is stored in the memory 25 of the pulse oximeter 2, is temporarily saved in the RAM 352 of the PC main body 30 via the oximeter I/F 27 and the PC I/F 351 (see FIG. 12).

Then, the measurement data downloaded to the PC 3 is analyzed by the SpO₂ calculator 31 and the tilt angle calculator 32 (Step S7). Specifically, the SpO₂ calculator 31 computes the number of times when the SpO₂ is lowered resulting from apnea of the subject H. Also, the tilt angle calculator 32 computes data concerning time-based change of the body angle of the subject H during his or her sleep.

Subsequently, the data synthesizer 331 of the main calculator 33 creates composite data concerning the SpO₂ curve and the time-based change of the body angle by expressing the data along a common time axis (Step S8). Then, the number of Dips with respect to each of the body angles is counted by the angle-based ODI calculator 333 to obtain angle-based ODI (Step S9).

Thereafter, a histogram showing a relation between body angle and the Dip number is created by the histogram calculator 336 so that a correlation is obtained between apnea and body position of the subject H in terms of angle information i.e. a body angle (Step S10). Then, the display controller 34 causes the display unit 37 to display or causes the output unit 38 to output the histogram as an image suitable to represent the histogram in response to receiving designation from the operating unit 36 (Step S11). Then, the routine ends. This is the flow on the entire operation of the system S. Next, flows of Step S3, Step S7, and Step S9 are described in detail one by one.

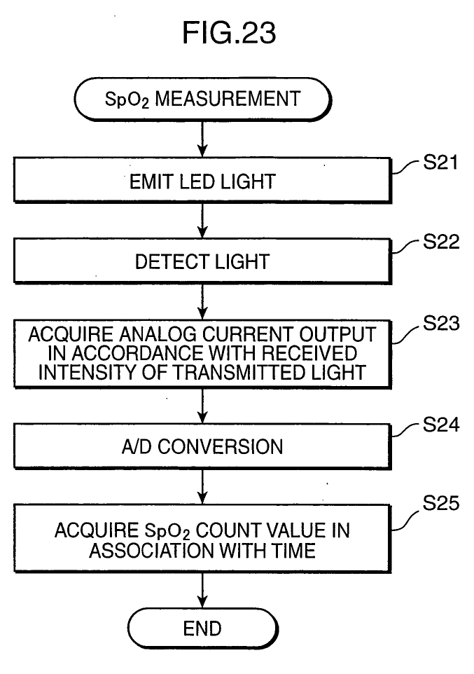

FIG. 23 is a flowchart showing details on the SpO₂ measurement in Step S3 of the flowchart in FIG. 22. When it is judged that the current time is coincident with the time of the predetermined sampling frequency, the red LED 211R or the infrared 211IR (see FIG. 4) provided in the probe 21 are turned on to emit red light or infrared light toward the finger F of the subject H (Step S21). The light detector 212 detects transmitted light through the finger F in synchronization with the light emission (Step S22), and an analog current output in accordance with the received light intensity is acquired by the light detecting circuit 212C (Step S23).

The acquired analog current output is converted into a digital measurement signal by the first A/D converter 231 (see FIG. 11) (Step S24). Then, the SpO₂ count detector 241 detects a count value of SpO₂ corresponding to the digital measurement signal at the predetermined sampling frequency (Step S25). The SpO₂ count value is stored in the measurement data storage 251 of the memory 25 in association with the time when the count value has been acquired. The above routine is cyclically repeated at the predetermined sampling frequency.

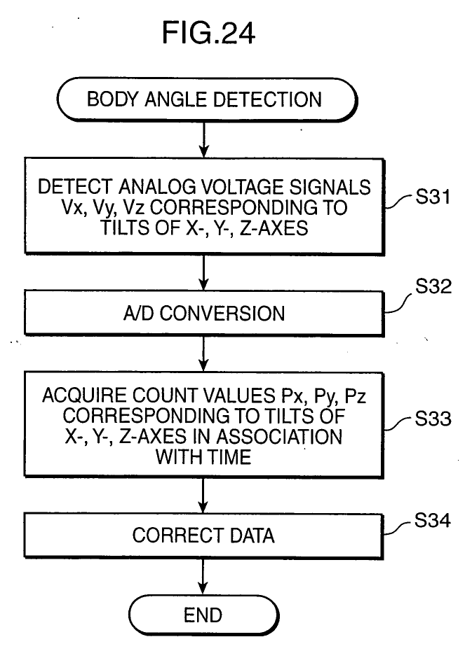

FIG. 24 is a flowchart showing details on the body angle detection in Step S3 of the flowchart in FIG. 22. When it is judged that the current time is coincident with the time of the sampling frequency, sensor outputs i.e. analog current signals regarding the X-, Y-, and Z-axes of the three-axis acceleration sensor 22 (see FIG. 11) are obtained. The analog current signals are current-to-voltage converted into voltage signals, which, in turn, are detected as analog voltage signals Vx, Vy, and Vz with respect to the X-, Y-, and Z-axes (Step S31).

The analog voltage signals, Vx, Vy, and Vz are analog-to-digitally converted into digital signals by the second A/D converter 232 (Step S32). Then, the body tilt count detector 243 detects count values Px, Py, and Pz corresponding to the respective tilts of the X-, Y-, and Z-axes, which correspond to the digital measurement signals obtained at the sampling frequency (Step S33).

Subsequently, the data corrector 243 corrects the count values Px, Py, and Pz with use of the correction amount count values X₀, Y₀, and Z₀, which are stored in the correction amount storage 252, to correct axial displacement resulting from attachment error of the three-axis acceleration sensor 22 to the pulse oximeter 2 (Step S34). The count values Px, Py, and Pz corresponding to the respective tilts of the X-, Y, and Z-axes after the data correction are stored in the measurement data storage 251 of the memory 25 in association with the time when the respective count values have been acquired. The above routine is cyclically repeated at the sampling frequency.

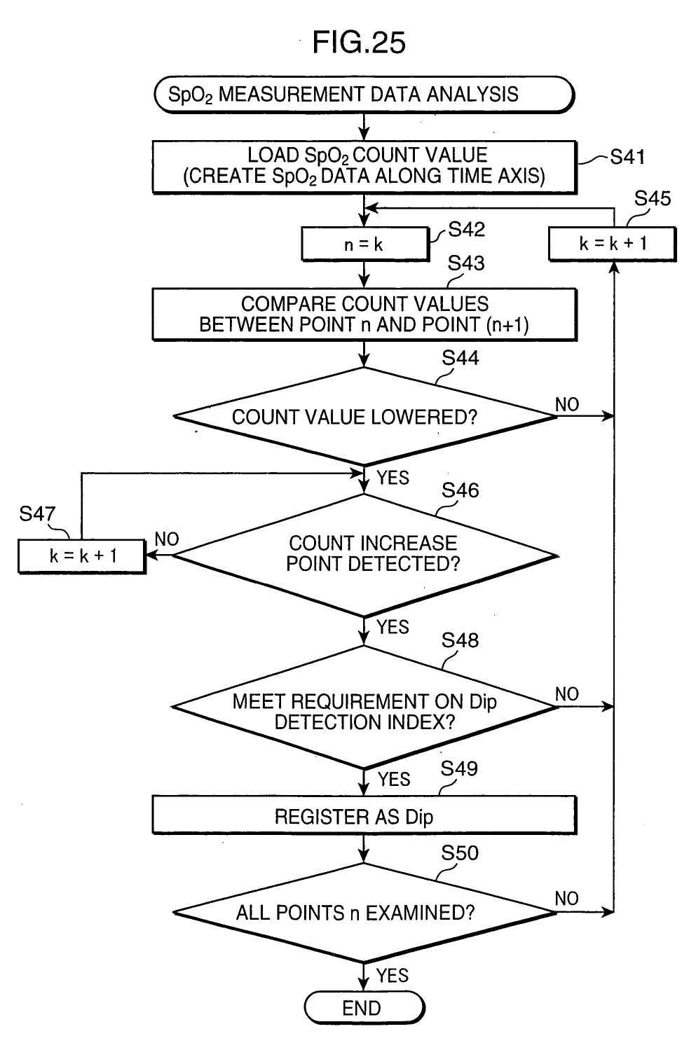

FIG. 25 is a flowchart showing details on the SpO₂ measurement data analysis in Step S7 of the flowchart in FIG. 22. First, the time series data generator 311 (see FIG. 12) of the SpO₂ calculator 31 creates data on a SpO₂ curve by expressing the SpO₂ count values which have been downloaded from the pulse oximeter 2 to the PC 3 along a time axis (Step S41). The data on the SpO₂ curve is data showing time-based change of SpO₂, as shown in FIG. 13, but actually is data that has been loaded to the RAM 352 or a like device.

A “significant Dip” is detected by executing the following operation regarding an SpO₂ count value at an arbitrary judging point n, wherein the judging point n is sequentially defined along the time axis e.g. at a time interval corresponding to the sampling frequency with respect to the SpO₂ curve obtained in Step S41 by the Dip detector 312. Specifically, at an initial stage of measurement, n=k (Step S42), and a comparison is made between SpO₂ count values between the judging point n and an adjacent point (n+1) (Step S43).

Then, a judgment is made whether the SpO₂ count value at the point (n+1) is lower than the SpO₂ count value at the point n (Step S44). If the SpO₂ count value at the point (n+1) is not lower than the SPO₂ count value at the point n (NO in Step S44), it means that there is no Dip. Accordingly, k is incremented by one: k=k+1 (Step S45), and the routine returns to Step S42 to cyclically repeat the above operation of setting a next point (n+1) on the time axis as a measurement reference.

If, on the other hand, the SpO₂ count value at the point (n+1) is lower than the SPO₂ count value at the point n (YES in Step S44), the point n is advanced on the time axis until a point where the SpO₂ count value is increased is found because there is a possibility that a Dip has occurred. Specifically, it is judged whether the SpO₂ count value is increased at the targeted point n after the start point of time when the candidate Dip is detected in Step S44 (Step S46). If it is judged that the SpO₂ count value is not increased after the start point of time when the candidate Dip is detected (NO in Step S46), k is incremented by one: k=k+1 to advance the point n (Step S47), and the judgment in Step S46 is cyclically repeated.

If, on the other hand, it is judged that the SpO₂ count value is increased (YES in Step S46), it means that the Dip is directed to an end. Accordingly, a judgment is made whether the candidate Dip satisfies the requirements on the aforementioned predetermined Dip detection index (Step S48). The Dip detection index may include a gradient of downslope of SpO₂ curve, a lowering degree of SpO₂, a time duration when lowering of SpO₂ is continued, or other phenomenon, as described above. In the case where the recovery time or recovery rate, i.e. a time or speed which is necessary for the SpO₂ count value to recover to its level corresponding to the point of time when the Dip is started to be observed is included as the index, a step is additionally provided after Step S46 to judge whether the SpO₂ count value is recovered to its level corresponding to the point of time when the Dip is started to be observed. Alternatively, the data may be smoothed by a moving-average method in judging whether the SpO₂ count value is decreased or increased.

If it is judged that the candidate Dip satisfies the requirements on the predetermined Dip detection index (YES in Step S48), the Dip detector 312 judges that the candidate Dip is a significant Dip, and registers the Dip in the RAM 352 or an equivalent device in association with time information relating to the time when the Dip is judged so (Step S49). In the registration, the judgment as to whether the Dip satisfies the requirements on the Dip detection index is executed based on the threshold information stored in the Dip threshold setter 313. For instance, if the lowering degree of SPO₂ is adopted as the index, the Dip detector 312 judges whether the candidate Dip satisfies the requirements on the lowering degree of SpO₂, using one or more than one of the thresholds Dip1, Dip2, and Dip3 shown in FIG. 13.

If, on the other hand, it is judged that the candidate Dip does not satisfy the requirements on the Dip detection index (NO in Step S48), k is incremented by one: k=k+1 to advance the point n (Step S45). Then, the routine returns to Step S42, and the operations from Step S42 to Step S48 are cyclically repeated to search for a next Dip. Also, in the case where it is judged that there remains a judging point n (NO in Step S50) after the registration of Dip in Step S49, similarly to the operation after the negative judgment in Step S48, the routine returns to Step S42 to cyclically repeat the operations to search for a next Dip. This is the operations on the SpO₂ measurement data analysis routine.

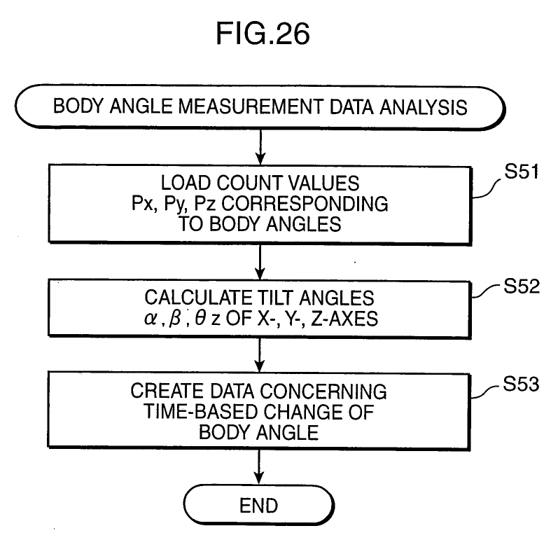

FIG. 26 is a flowchart showing details on the body angle measurement data analysis in Step S7 of the flowchart in FIG. 22. First, data concerning the count values Px, Py, and Pz corresponding to the respective tilts of the X-, Y-, and Z-axes that has been downloaded from the oximeter 2 to the PC 3 are loaded to the RAM 352 or an equivalent device (Step S51). The data is data showing time-based change of the count values Px, Py, and Pz corresponding to the tilts of the X-, Y-, and Z-axes.

Referring to FIG. 26, the X-axis tilt angle detector 321, the Y-axis tilt angle detector 322, and the Z-axis tilt angle detector 323 of the tilt angle calculator 32 respectively calculate tilt angles α, β, and θz of the X-, Y-, and Z-axes (Step S52). Then, the tilt angle data generator 324 detects a time period when the subject H is in a seated position based on the time-based change of the tilt angle α of the X-axis, and obtains data concerning time-based change of the body angle of the subject H based on the tilt angle β of the Y-axis and the tilt angle θz of the Z-axis (Step S53). The data is as shown in FIG. 15, for instance. This is the operations on the body angle measurement data analysis routine.

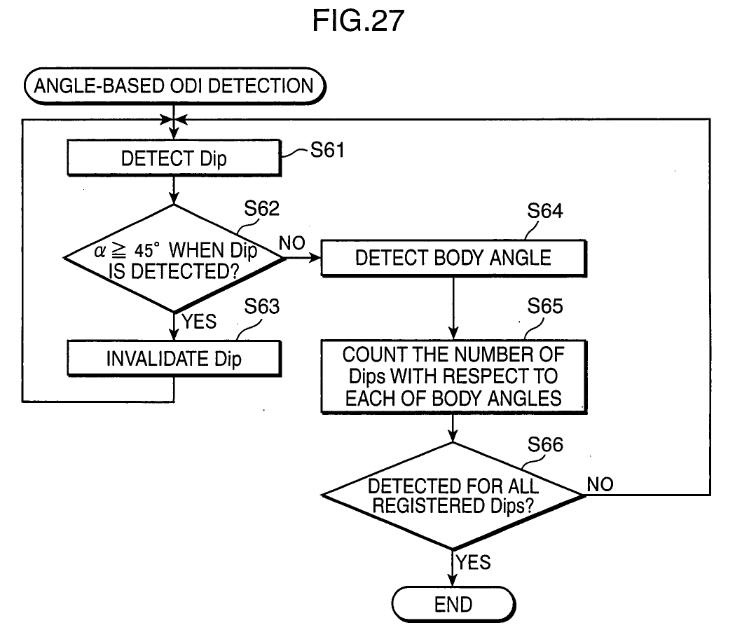

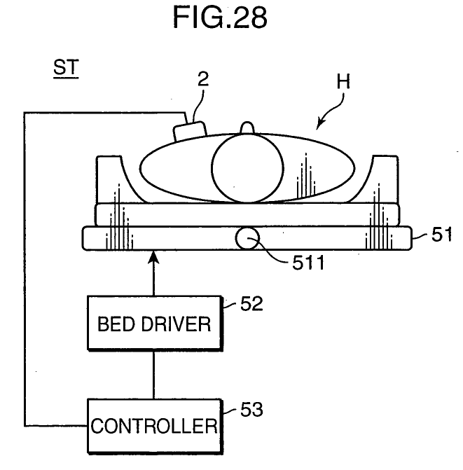

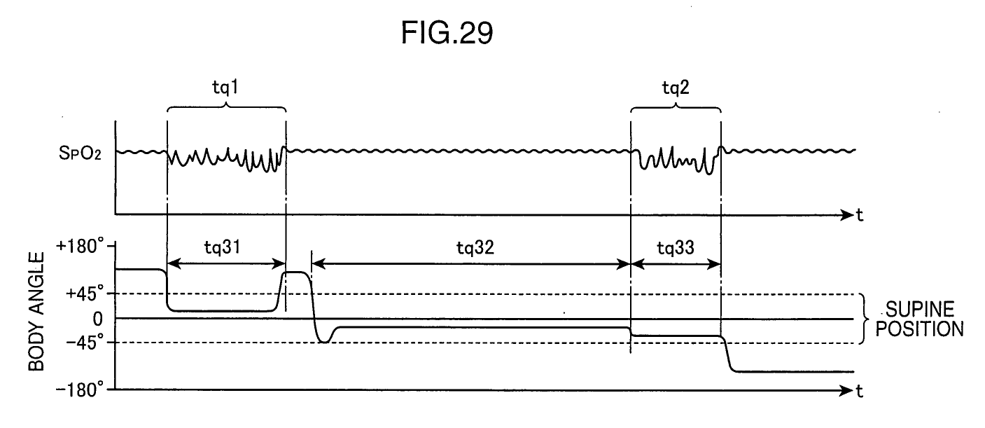

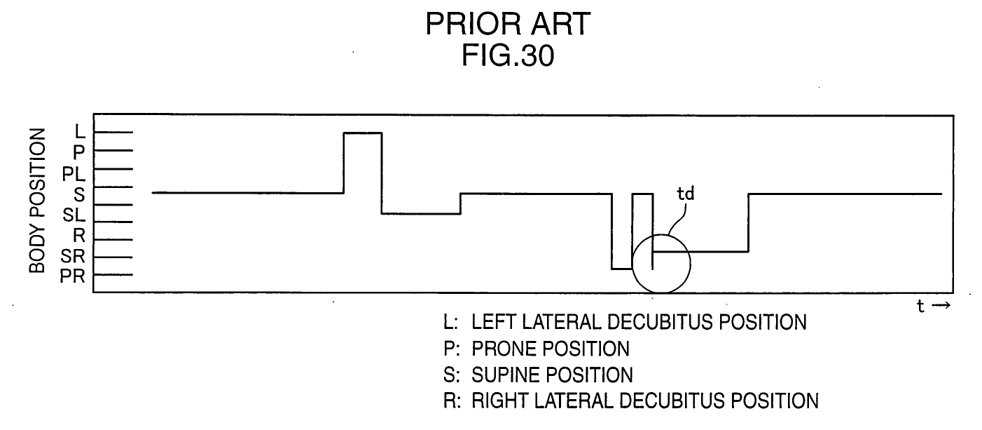

FIG. 27 is a flowchart showing details on the angle-based ODI detection in Step S9 of the flowchart in FIG. 22. In this operation, the number of Dips with respect to each of the body angles is counted in association with the data concerning time-based change of the body angle obtained by implementing the operations of the flowchart in FIG. 26. The Dips are detected and registered in accordance with the operations of the flowchart in FIG. 25.

Referring to FIG. 27, first, the Dip that has been registered and stored at an earliest time in the RAM 352 is retrieved, for instance (Step S61). Then, data discrimination is conducted by the data discriminator 332 of the main calculator 33, in other words, it is judged whether the tilt angle α of the X-axis when the Dip is detected is 45° or larger (Step S62). If α≧45° (YES in Step S62), the judgment result means that the Dip is invalid because it indicates that the subject H is in a seated position. Accordingly, the Dip is invalidated (Step S63), and the routine returns to Step S61 to detect a second earliest Dip that has been registered and stored in the RAM 352.

If, on the other hand, α<45° (NO in Step S62), the angle-based ODI calculator 333 detects a body angle of the subject H when the Dip is detected based on the tilt angle β of the Y-axis and the tilt angle θz of the Z-axis (Step S64). Then, the number of Dips is counted with respect to each of the predetermined body angles, e.g., by 5° interval (Step S65). Then, it is judged whether there remains any Dip that has been registered in the RAM 352 (Step S66). If it is judged that there remains a Dip (NO in Step S66), the routine returns to Step S61 to detect a next Dip. The above operations are cyclically repeated with respect to all the registered Dips. Angle-based AHI may be detected by implementing operations similar to the operations of the flowchart in FIG. 27.

(Description on Modifications)

In the embodiment, described is the sleep evaluation system S of evaluating a correlation between apnea and body angle of a subject to diagnose SAS dedicatedly. The embodiment may be modified to provide a sleep support system of providing a subject i.e. a SAS patient with a comfortable sleep environment with no or less likelihood of apnea.

FIG. 28 is a simplified block diagram showing an arrangement of the sleep support system ST. The sleep support system ST includes a pulse oximeter 2, serving as a measuring device, which is capable of concurrently measuring a body angle and a blood oxygen saturation of a subject H at a predetermined sampling frequency, a bed 51 designed in such a manner that the angle of the bed 51 is pivotally adjustable about an axis 511 of rotation while supporting the subject H in a certain lying position, a bed driver 52 for drivingly adjusting the angle of the bed 51, and a controller 53 for determining the angle of the bed 51 adjusted by the bed driver 52.

A measurement operation of the pulse oximeter 2 is controlled by the controller 53 so that measurement data concerning the body angle and the blood oxygen saturation of the subject H can be obtained at a predetermined sampling frequency. The measurement data is sent to the controller 53 in a time-series manner. The measurement data is stored by an amount corresponding to a time period required for diagnosing apnea of the subject H. The controller 53 evaluates a correlation between apnea based on lowering of the blood oxygen saturation, and body angle of the subject H by expressing the acquired measurement data along a time axis to detect a body angle with no or less likelihood of apnea for the subject H.

Upon detecting the recommended body angle with no or less likelihood of apnea, the controller 53 sends, to the bed driver 52, a control signal to position the bed 51 to such an angle that makes it possible to secure the subject H at the recommended body angle. In response to receiving the control signal, the bed driver 52 drives the bed 51 so that the bed 51 is positioned to the angle to secure the subject H at the recommended body angle. There is likelihood that the subject H may roll out of the bed 51 if the angle of the bed 51 is too large. In view of this, the top surface of the bed 51 has a certain concave portion in the Y-direction (see FIG. 9), as shown in FIG. 28. Also, a predetermined upper limit is defined for the angle of the bed 51 so that the bed driver 52 does not tilt the bed 51 over the upper limit. In this arrangement, a comfortable sleep environment is provided for the subject H, with the body position of the subject H secured to such an angle that is unlikely or less likely to cause apnea.

Further, it is possible to provide an operation program of executing a process to be implemented by the sleep evaluation system S, as an embodiment to carry out the invention. The program may be provided as a program product by recording the program on a computer-readable recording medium, which is an attachment to a computer, such as a flexible disk, a CD-ROM, an ROM, an RAM, or a memory card. Also, the program may be provided by recording the program on a recording medium equipped in the PC main body 30 shown in FIG. 2. Further alternatively, the program may be provided by downloading via a network.

In general, the routines executed to implement the embodiment of the invention, whether implemented as part of an operating system or a specific application, component, program, object, module or sequence of instructions will be referred to as “programs”. The program comprises one or more instructions that are resident at various times in various memory and storage devices in a computer, and that cause the computer to perform the steps necessary to execute steps or elements embodying the various aspects of the invention.

The embodiment of the invention has and will be described in the context of functioning the computer and computer system. However, those skilled in the art will appreciate that various embodiments of the invention are capable of being distributed as a program product in a variety of forms, and that the invention applies equally regardless of the particular type of signal bearing media used to actually carry out the distribution. Examples of signal bearing media include but are not limited to recordable type media such as volatile and non-volatile memory devices, floppy and other removable disks, hard disk drives, optical disks (e.g., CD-ROM's, DVD's, etc.), among others, and transmission type media such as digital and analog communication links, including the Internet.

As described above, a sleep evaluation method is performed by measuring a body angle and a blood oxygen saturation of a subject in sleep concurrently at a predetermined sampling frequency to acquire measurement data concerning the body angle and the blood oxygen saturation; and expressing the measurement data along a time axis to evaluate a correlation between apnea based on lowering of the blood oxygen saturation and the body angle of the subject.

With this method, the body position of the subject in sleep can be measured in terms of body angle, which is a minute parameter, in place of body direction, which is a rough parameter, such as supine position or lateral decubitus position, and the blood oxygen saturation can be concurrently measured with the body angle. Accordingly, a correlation between apnea and body position of the subject in sleep can be accurately obtained.

The correlation between apnea and body position of the subject is obtained by way of a relation between apnea and body angle, which represents how much the body of the subject is tilted with respect to a reference plane in terms of angle, in place of a relation between apnea and body direction, which is a rough parameter as detected in the conventional art. This enables to securely acquire a causal relation between apnea and body position of the subject. Accordingly, the subject i.e. individual SAS patients are provided with a proper treatment having dependence on body position.

A sleep evaluation system comprises: an evaluation parameter detector for measuring an evaluation parameter of a subject, the evaluation parameter being varied due to sleep apnea of the subject; a body position detector for detecting a body position of the subject in terms of angle information; a storage for storing measurement data acquired by the evaluation parameter detector and by the body position detector therein; and a controller for causing the evaluation parameter detector to measure the evaluation parameter, and causing the body position detector to measure a body angle of the subject at a predetermined sampling frequency to store the measurement data in the storage.

With this arrangement, the body position of the subject in sleep can be measured by the body position detector in terms of angle information, the evaluation parameter, which is varied due to sleep apnea of the subject, can be measured by the evaluation parameter detector, and the measurement data concerning the body angle and the evaluation parameter can be stored in the storage. This arrangement enables to securely acquire the correlation between body position and evaluation parameter of the subject in sleep based on the measurement data stored in the storage.

The correlation between body position and evaluation parameter of the subject in sleep can be accurately obtained based on the measurement data having relevancy to apnea, which is stored in the storage. This arrangement enables to provide the individual SAS patients with a proper treatment having dependence on body position.

Preferably, the evaluation parameter detector may include a sensor for detecting a blood oxygen saturation of the subject.

With the above arrangement, the correlation between body position of the subject in sleep and apnea, which is observed as a variation in blood oxygen saturation, e.g., arterial blood oxygen saturation can be accurately obtained. Since the blood oxygen saturation is used as the evaluation parameter having relevancy to apnea, the evaluation parameter can be obtained with use of a commercially available pulse oximeter or a like device non-invasively with less stress to the subject.

Preferably, the sleep evaluation system may be further provided with an analyzer for analyzing a correlation between a variation in the evaluation parameter or in a blood oxygen saturation, and the body angle of the subject based on the measurement data stored in the storage to determine a correlation between apnea and the body angle of the subject.

With this arrangement, a causal relation between apnea and body angle of the subject in sleep can be obtained with use of the analyzer. According to the arrangement, ODI, which is an index for determining the degree of severity of SAS, can be obtained with respect to each of the body angles for individual subjects with use of the analyzer.

The analyzer may preferably analyze a correlation at least between measurement data concerning an airflow of a respiratory system and movements of a body trunk portion of the subject, and the body angle of the subject, in addition to the correlation between the variation in the blood oxygen saturation and the body angle of the subject to determine a correlation between the apnea or a low respiration, and the body angle of the subject.

With this arrangement, a causal relation between apnea or low respiration, and body angle of the subject in sleep can be obtained with use of the analyzer. According to the arrangement, AHI, which is a frequently used index among medical institutes with use of PSG, can be obtained with respect to each of the body angles for individual subjects with use of the analyzer.

It may be preferable that the body position detector judges at least whether the body position of the subject is a seated position or a lying position, and the storage stores information relating to the judgment result on the body position of the subject therein.

This construction enables to discriminate a period corresponding to a state that the subject wakes up in the middle of sleep evaluation sometimes accompanied by walking, which normally includes a transient time corresponding to a seated position, thereby enabling to discriminate the measurement data acquired while the subject is in the seated position from the measurement data acquired while the subject is in the lying position. Since the measurement data acquired while the subject is in the seated position, which cannot be handled as valid measurement data can be discriminated from the measurement data acquired while the subject is in the lying position, reliability on measurement data can be enhanced.

It may be preferable that the body position detector includes a three-axis acceleration sensor having a first axis, a second axis, and a third axis, an output from the three-axis acceleration sensor with respect to the first axis is used to measure the body angle of the subject, an output from the three-axis acceleration sensor with respect to the second axis is used to judge whether the body position of the subject is the seated position or the lying position, and an output from the three-axis acceleration sensor with respect to the third axis is used to judge whether the lying position of the subject is a supine position or a prone position.

With this arrangement, the body angle of the subject can be measured in different body positions, i.e., a supine position and a prone position with use of the single sensing device, and a judgment can be made as to whether the subject is in the seated position. According to this arrangement, attaching the single sensing device, i.e., the three-axis acceleration sensor to the subject enables to measure the body angle of the subject in different body positions, i.e., the supine position and the prone position, and enables to judge whether the subject is in the seated position. This arrangement enables to reduce a stress to the subject during the measurement. Also, the configuration of the system can be simplified by incorporating the three-axis acceleration sensor in the pulse oximeter or a like device.

Preferably, the analyzer may analyze a correlation between a variation in the evaluation parameter or in a blood oxygen saturation, and the body angle of the subject, using measurement data indicating that the body position of the subject is the lying position.

With this arrangement, sleep of the subject can be evaluated, with the invalid data acquired while the subject is in the seated position eliminated, with use of the analyzer. Accordingly, the correlation between apnea and body angle of the subject can be more accurately obtained.

A sleep evaluation system comprises: a pulse oximeter including a blood oxygen saturation measuring device for acquiring measurement data concerning a blood oxygen saturation of a subject, a body position detector for acquiring measurement data concerning a body angle of the subject, a storage for storing the measurement data acquired by the blood oxygen saturation measuring device and by the body position detector therein, and a controller for causing the blood oxygen saturation measuring device to acquire the measurement data concerning the blood oxygen saturation of the subject, and causing the body position detector to acquire the measurement data concerning the body angle of the subject at a predetermined sampling frequency to store the measurement data acquired by the blood oxygen saturation measuring device and by the body position detector in the storage; a fastening device for securely holding the body position detector of the pulse oximeter on a body trunk portion of the subject; and a processor for acquiring the measurement data stored in the storage of the pulse oximeter to analyze a correlation between a variation in the blood oxygen saturation and the body angle of the subject for display.

With this arrangement, the pulse oximeter is provided with the blood oxygen saturation measuring device for acquiring the measurement data concerning the blood oxygen saturation, and the body tilt detector for acquiring the measurement data concerning the body angle. The measurement data is acquired with the pulse oximeter attached to the body trunk portion of the subject, and the acquired measurement data is stored in the storage of the pulse oximeter. The measurement data stored in the storage is read out after the subject wakes up, and the readout measurement data is analyzed by the processor such as a personal computer. Thus, a causal relation between apnea and body angle of the subject in sleep can be obtained.

The system can be configured by the pulse oximeter, the personal computer, and the like, the measurement data concerning the body angle and the blood oxygen saturation of the subject can be acquired with use of the pulse oximeter alone, and the measurement data can be stored in the storage. This arrangement enables to simplify the configuration of the system and reduce a stress to the subject during the measurement.

Preferably, the processor may include a display controller for displaying a correlation between a peak where the blood oxygen saturation is lowered and the body angle of the subject by expressing the measurement data along a time axis.

With this arrangement, the correlation between apnea and body angle of the subject can be securely obtained based on the relation between the peak where the blood oxygen saturation is lowered and the body angle of the subject. The correlation between apnea and body angle of the subject can be securely obtained by observing an image or the like generated and displayed by the display controller.

Preferably, the processor may include a histogram calculator for expressing a correlation between a frequency of occurrence of a peak where the blood oxygen saturation is lowered and the body angle of the subject in a histogram.

With this arrangement, since the correlation between the frequency of occurrence of apnea and the body angle of the subject can be securely obtained, ODI or a like index can be automatically displayed with respect to each of the body angles for individual subjects.

Preferably, the processor may include a calculator for outputting a body angle of the subject with no or less likelihood of occurrence of the peak where the blood oxygen saturation is lowered as a recommended body angle for the subject with no or less likelihood of apnea.

With this arrangement, since the body angle capable of preventing apnea of the subject can be readily obtained, an approach for treating individual SAS patients can be readily determined.

It may be preferable that the body position detector of the pulse oximeter judges at least whether a body position of the subject is a seated position or a lying position, the storage stores information relating to the judgment result on the body position therein, and the processor includes a data discriminator for discriminating measurement data indicating that the body position of the subject is the lying position.

With this arrangement, since sleep of the subject can be evaluated with use of the processor, with the invalid data acquired while the subject is in the seated position being discriminated and eliminated by the data discriminator, the correlation between apnea and body angle of the subject can be more accurately determined.

A pulse oximeter comprises: a blood oxygen saturation measuring device for acquiring measurement data concerning a blood oxygen saturation of a subject; a body position detector for acquiring measurement data concerning a body angle of the subject; a storage for storing the measurement data acquired by the blood oxygen saturation measuring device and by the body position detector therein; and a controller for causing the blood oxygen saturation measuring device to acquire the measurement data concerning the blood oxygen saturation of the subject, and causing the body position detector to acquire the measurement data concerning the body angle of the subject at a predetermined sampling frequency to store the measurement data acquired by the blood oxygen saturation measuring device and by the body position detector in the storage.

With this arrangement, the measurement data concerning the blood oxygen saturation and the measurement data concerning the body angle of the subject in sleep can be acquired with use of the pulse oximeter, and stored in the storage. Accordingly, the sleep evaluation system for obtaining a causal relation between apnea and body angle of the subject in sleep can be configured by causing the processor such as a personal computer to read out the measurement data stored in the storage for analysis after the subject wakes up. The causal relation between apnea and body angle of the subject can be obtained by reading out the measurement data from the storage and analyzing the readout measurement data after the subject wakes up. Thus, the system for evaluating dependence of SAS patients on body position can be configured with use of the pulse oximeter alone.

Preferably, the pulse oximeter may be further provided with a display unit and a processor. The processor has a function of acquiring the measurement data stored in the storage, and analyzing a correlation between a variation in the blood oxygen saturation, and the body angle of the subject to cause the display unit to display the correlation.

With this arrangement, since the causal relation between apnea and body angle of the subject in sleep can be obtained with use of the pulse oximeter alone without use of an external processor, the configuration of the system for evaluating dependence of SAS patients on body position can be simplified.

Preferably, the pulse oximeter may be further provided with a direction guide provided on an outer surface of a casing of the pulse oximeter to notify a direction in which the pulse oximeter is normally attached to the subject.

With this arrangement, the subject is guided to attach the pulse oximeter in a proper direction as displayed on the guide display. The subject is securely guided to attach the pulse oximeter in a proper direction. Accordingly, in the case where the three-axis acceleration sensor is used as the body position detector, for instance, outputs from the three-axis acceleration sensor with respect to the three axes can be used as designed. If the pulse oximeter is attached in a wrong direction, rolling over of the subject in a rightward direction may be misjudged as rolling over in a leftward direction. Providing the direction guide enables to prevent occurrence of such a misjudgment.

A program product is adapted for operating a sleep evaluation system provided with an evaluation parameter detector for measuring an evaluation parameter of a subject, the evaluation parameter being varied due to sleep apnea of the subject, a body position detector for detecting a body position of the subject in terms of angle information, a storage for storing measurement data acquired by the evaluation parameter detector and by the body position detector therein, and an analyzer. The program product comprises: a program which allows a computer to execute the steps of making the evaluation parameter detector to measure the evaluation parameter, and making the body position detector to measure a body angle of the subject at a predetermined sampling frequency to acquire measurement data concerning the evaluation parameter and the body angle, making the storage to store the measurement data therein, and making the analyzer to analyze a correlation between a variation in the evaluation parameter and the body angle of the subject based on the measurement data stored in the storage; and a signal bearing media bearing the program.

With this program product, the correlation between body position and evaluation parameter of the subject in sleep can be evaluated with use of the analyzer based on the measurement data having relevancy to apnea of the subject, which is stored in the storage. This enables to provide individual SAS patients with a proper treatment having dependence on body position.

Another program product is adapted for operating a sleep evaluation system provided with a pulse oximeter including a blood oxygen saturation measuring device for acquiring measurement data concerning a blood oxygen saturation of a subject, a body position detector for acquiring measurement data concerning a body angle of the subject, a storage for storing the measurement data acquired by the blood oxygen saturation measuring device and by the body position detector therein, and a processor. The program product comprises: a program which allows a computer to execute the steps of making the blood oxygen saturation measuring device to acquire measurement data concerning the blood oxygen saturation, and making the body position detector to acquire measurement data concerning the body angle at a predetermined sampling frequency, making the storage to store the measurement data therein, and making the processor to acquire the measurement data stored in the storage of the pulse oximeter to analyze a correlation between a variation in the blood oxygen saturation and the body angle of the subject; and a signal bearing media bearing the program.

With this program product, the correlation between apnea and body position of the subject in sleep can be evaluated with use of the processor based on the measurement data concerning the blood oxygen saturation and the body angle, which is stored in the storage of the pulse oximeter, by using the system comprised of the pulse oximeter and the processor such as a personal computer. This enables to provide individual SAS patients with a proper treatment having dependence on body position.

A sleep support system comprises: a bed for supporting a subject in a lying position; a bed driver for adjusting an angle of the bed; and a controller for determining the angle of the bed adjusted by the bed driver. The controller is operative to cause a measuring device to measure a body angle and a blood oxygen saturation of the subject in sleep on the bed concurrently at a predetermined sampling frequency to acquire measurement data concerning the body angle and the blood oxygen saturation, to evaluate a correlation between apnea based on lowering of the blood oxygen saturation and the body angle of the subject by expressing the measurement data along a time axis, to determine a body angle of the subject with no or less likelihood of apnea, and to output the body angle of the subject with no or less likelihood of apnea as a designated angle of the bed to be adjusted by the bed driver.

With this arrangement, the angle of the bed with no or less likelihood of apnea can be determined concurrently with detection of the body angle where apnea of the subject is observed. This arrangement enables to provide the subject with a comfortable sleep environment with no or less likelihood of apnea. Since the angle of the bed is adjusted so that the body of the subject is secured to such a body position that has no or less likelihood of apnea, while detecting the apnea, a comfortable sleep environment is provided for the subject.

Although the present invention has been fully described by way of example with reference to the accompanying drawings, it is to be understood that various changes and modifications will be apparent to those skilled in the art. Therefore, unless otherwise such changes and modifications depart from the scope of the present invention hereinafter defined, they should be construed as being included therein.

What is claimed is:

1. A sleep evaluation method comprising the steps of:
measuring a body angle and a blood oxygen saturation of a subject in sleep concurrently at a predetermined sampling frequency to acquire measurement data concerning the body angle and the blood oxygen saturation; and
expressing the measurement data along a time axis to evaluate a correlation between apnea based on lowering of the blood oxygen saturation and the body angle of the subject.

2. A sleep evaluation system comprising:
an evaluation parameter detector for measuring an evaluation parameter of a subject, the evaluation parameter being varied due to sleep apnea of the subject;
a body position detector for detecting a body position of the subject in terms of angle information;
a storage for storing measurement data acquired by the evaluation parameter detector and by the body position detector therein; and
a controller for causing the evaluation parameter detector to measure the evaluation parameter, and causing the body position detector to measure a body angle of the subject at a predetermined sampling frequency to store the measurement data in the storage.

3. The sleep evaluation system according to claim 2, wherein
the evaluation parameter detector includes a sensor for detecting a blood oxygen saturation of the subject.

4. The sleep evaluation system according to claim 2, further comprising an analyzer for analyzing a correlation between a variation in the evaluation parameter or in a blood oxygen saturation, and the body angle of the subject based on the measurement data stored in the storage to determine a correlation between apnea and the body angle of the subject.

5. The sleep evaluation system according to claim 4, wherein
the analyzer analyzes a correlation at least between measurement data concerning an airflow of a respiratory system and movements of a body trunk portion of the subject, and the body angle of the subject, in addition to the correlation between the variation in the blood oxygen saturation and the body angle of the subject to determine a correlation between the apnea or a low respiration, and the body angle of the subject.

6. The sleep evaluation system according to claim 2, wherein
the body position detector judges at least whether the body position of the subject is a seated position or a lying position, and
the storage stores information relating to the judgment result on the body position of the subject therein.

7. The sleep evaluation system according to claim 6, wherein
the body position detector includes a three-axis acceleration sensor having a first axis, a second axis, and a third axis,
an output from the three-axis acceleration sensor with respect to the first axis is used to measure the body angle of the subject,
an output from the three-axis acceleration sensor with respect to the second axis is used to judge whether the body position of the subject is the seated position or the lying position, and
an output from the three-axis acceleration sensor with respect to the third axis is used to judge whether the lying position of the subject is a supine position or a prone position.

8. The sleep evaluation system according to claim 6, wherein
the analyzer analyzes a correlation between a variation in the evaluation parameter or in a blood oxygen saturation, and the body angle of the subject, using measurement data indicating that the body position of the subject is the lying position.

9. A sleep evaluation system comprising:
a pulse oximeter including
a blood oxygen saturation measuring device for acquiring measurement data concerning a blood oxygen saturation of a subject,
a body position detector for acquiring measurement data concerning a body angle of the subject,
a storage for storing the measurement data acquired by the blood oxygen saturation measuring device and by the body position detector therein, and
a controller for causing the blood oxygen saturation measuring device to acquire the measurement data concerning the blood oxygen saturation of the subject, and causing the body position detector to acquire the measurement data concerning the body angle of the subject at a predetermined sampling frequency to store the measurement data acquired by the blood oxygen saturation measuring device and by the body position detector in the storage;
a fastening device for securely holding the body position detector of the pulse oximeter on a body trunk portion of the subject; and
a processor for acquiring the measurement data stored in the storage of the pulse oximeter to analyze a correlation between a variation in the blood oxygen saturation and the body angle of the subject for display.

10. The sleep evaluation system according to claim 9, wherein
the processor includes a display controller for displaying a correlation between a peak where the blood oxygen saturation is lowered and the body angle of the subject by expressing the measurement data along a time axis.

11. The sleep evaluation system according to claim 9, wherein
the processor includes a histogram calculator for expressing a correlation between a frequency of occurrence of a peak where the blood oxygen saturation is lowered and the body angle of the subject in a histogram.

12. The sleep evaluation system according to claim 11, wherein
the processor includes a calculator for outputting a body angle of the subject with no or less likelihood of occurrence of the peak where the blood oxygen saturation is lowered as a recommended body angle for the subject with no or less likelihood of apnea.

13. The sleep evaluation system according to claim 9, wherein
the body position detector of the pulse oximeter judges at least whether a body position of the subject is a seated position or a lying position,
the storage stores information relating to the judgment result on the body position therein, and
the processor includes a data discriminator for discriminating measurement data indicating that the body position of the subject is the lying position.

14. A pulse oximeter comprising:
a blood oxygen saturation measuring device for acquiring measurement data concerning a blood oxygen saturation of a subject;
a body position detector for acquiring measurement data concerning a body angle of the subject;
a storage for storing the measurement data acquired by the blood oxygen saturation measuring device and by the body position detector therein; and
a controller for causing the blood oxygen saturation measuring device to acquire the measurement data concerning the blood oxygen saturation of the subject, and causing the body position detector to acquire the measurement data concerning the body angle of the subject at a predetermined sampling frequency to store the measurement data acquired by the blood oxygen saturation measuring device and by the body position detector in the storage.

15. The pulse oximeter according to claim 14, further comprising a display unit and a processor, wherein
the processor has a function of acquiring the measurement data stored in the storage, and analyzing a correlation between a variation in the blood oxygen saturation, and the body angle of the subject to cause the display unit to display the correlation.

16. The pulse oximeter according to claim 14, further comprising a direction guide provided on an outer surface of a casing of the pulse oximeter to notify a direction in which the pulse oximeter is normally attached to the subject.

17. A program product for operating a sleep evaluation system provided with an evaluation parameter detector for measuring an evaluation parameter of a subject, the evaluation parameter being varied due to sleep apnea of the subject, a body position detector for detecting a body position of the subject in terms of angle information, a storage for storing measurement data acquired by the evaluation parameter detector and by the body position detector therein, and an analyzer, the program product comprising:
a program which allows a computer to execute the steps of
making the evaluation parameter detector to measure the evaluation parameter, and making the body position detector to measure a body angle of the subject at a predetermined sampling frequency to acquire measurement data concerning the evaluation parameter and the body angle,
making the storage to store the measurement data therein, and
making the analyzer to analyze a correlation between a variation in the evaluation parameter and the body angle of the subject based on the measurement data stored in the storage; and
a signal bearing media bearing the program.

18. A program product for operating a sleep evaluation system provided with a pulse oximeter including a blood oxygen saturation measuring device for acquiring measurement data concerning a blood oxygen saturation of a subject, a body position detector for acquiring measurement data concerning a body angle of the subject, a storage for storing the measurement data acquired by the blood oxygen saturation measuring device and by the body position detector therein, and a processor, the program product comprising:
a program which allows a computer to execute the steps of
making the blood oxygen saturation measuring device to acquire measurement data concerning the blood oxygen saturation, and making the body position detector to acquire measurement data concerning the body angle at a predetermined sampling frequency,
making the storage to store the measurement data therein, and
making the processor to acquire the measurement data stored in the storage of the pulse oximeter to analyze a correlation between a variation in the blood oxygen saturation and the body angle of the subject; and
a signal bearing media bearing the program.

19. A sleep support system comprising:
a bed for supporting a subject in a lying position;
a bed driver for adjusting an angle of the bed; and
a controller for determining the angle of the bed adjusted by the bed driver,
the controller being operative to cause a measuring device to measure a body angle and a blood oxygen saturation of the subject in sleep on the bed concurrently at a predetermined sampling frequency to acquire measurement data concerning the body angle and the blood oxygen saturation, to evaluate a correlation between apnea based on lowering of the blood oxygen saturation and the body angle of the subject by expressing the measurement data along a time axis, to determine a body angle of the subject with no or less likelihood of apnea, and to output the body angle of the subject with no or less likelihood of apnea as a designated angle of the bed to be adjusted by the bed driver.
